(19) United States
(12) Patent Application Publication
(10) Pub. No.: US 2024/0376451 A1
(43) Pub. Date: Nov. 14, 2024

(54) MAD NUCLEASES

(71) Applicant: Inscripta, Inc., Pleasanton, CA (US)

(72) Inventors: Juhan KIM, Pleasanton, CA (US); Benjamin MIJTS, Pleasanton, CA (US); Aamir MIR, Pleasanton, CA (US)

(73) Assignee: Inscripta, Inc., Pleasanton, CA (US)

(21) Appl. No.: 18/348,128

(22) PCT Filed: Jan. 3, 2022
(86) PCT No.: PCT/US2022/011052

§ 371 Date: Jul. 6, 2023

Related U.S. Application Data
(60) Provisional application No. 63/134,938, filed on Jan. 7, 2021.

(51) Int. Cl. C12N 9/22; C12N 15/11

(52) U.S. Cl. CPC C12N 9/22; C12N 15/11; C12N 2310/20

(57) ABSTRACT

The present disclosure provides new RNA-guided nucleases for making rational, direct edits to nucleic acids in live cells; specifically, the present disclosure provides Type V MAD nucleases (e.g., RNA-guided nucleases or RGNs) with altered PAM preferences and/or altered activity at different temperatures or fidelity, and/or varied nuclease activities; all changes that may increase the versatility of a nucleic acid-guided nuclease for certain editing tasks.

### RELATED CASES

This International PCT application claims priority to U.S. Ser. No. 63/134,938, filed 7 Jan. 2021, entitled “MAD NUCLEASES”, which is incorporated by reference in its entirety.

### FIELD OF THE INVENTION

This invention relates to new Type V nucleic acid-guided nucleases for making rational, directed edits to live cells.

### INCORPORATION BY REFERENCE

Submitted with the present application is an electronically filed sequence listing via EFS-Web as an ASCII formatted sequence listing, entitled “INSC084US_seq_list_20211229”, created Dec. 29, 2021, and 99,000 bytes in size. The sequence listing is part of the specification filed herewith and is incorporated by reference in its entirety.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow manipulation of gene sequence; hence, gene function. These nucleases include nucleic acid-guided nucleases. The range of target sequences that nucleic acid-guided nucleases can recognize, however, is constrained by the need for a specific PAM to be located near the desired target sequence. PAMs are short nucleotide sequences recognized by a gRNA/nuclease complex where this complex directs editing of the target sequence. The precise PAM sequence and PAM length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering nucleic acid-guided nucleases or mining for new nucleic acid-guided nucleases may provide nucleases with altered PAM preferences and/or altered activity at different temperatures or fidelity, and/or varied nuclease activities; all changes that may increase the versatility of a nucleic acid-guided nuclease for certain editing tasks.

There is thus a need in the art of nucleic acid-guided nuclease gene editing for novel nucleases with varied properties. The novel MAD nucleases described herein satisfy this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides Type V MAD nucleases (e.g., RNA-guided nucleases or RGNs) with varied PAM preferences, and/or varied activity in mammalian cells.

Thus, in one embodiment there are provided Type V MAD nuclease systems that perform nucleic acid-guided nuclease editing including a MAD293 system comprising SEQ ID NOs: 1 (MAD293 nuclease), 2 (CRISPR RNA) and 3 (trans-activating crispr RNA); a MAD294 system comprising SEQ ID NOs: 4 (MAD294 nuclease), 5 (CRISPR RNA) and 6 (trans-activating crispr RNA); a MAD295 system comprising SEQ ID NOs: 7 (MAD295 nuclease), 8 (CRISPR RNA) and 9 (trans-activating crispr RNA); a MAD296 system comprising SEQ ID NOs: 10 (MAD296 nuclease), 11 (CRISPR RNA) and 12 (trans-activating crispr RNA); a MAD297 system comprising SEQ ID NOs: 13 (MAD297 nuclease), 14 (CRISPR RNA) and 15 (trans-activating crispr RNA); a MAD298 system comprising SEQ ID NOs: 16 (MAD298 nuclease), 17 (CRISPR RNA) and 18 (trans-activating crispr RNA); and a MAD299 system comprising SEQ ID NOs: 19 (MAD299 nuclease), 20 (CRISPR RNA) and 21 (trans-activating crispr RNA). In some aspects, the MAD system components are delivered as sequences to be transcribed (in the case of the gRNA components) and transcribed and translated (in the case of the MAD nuclease), and in some aspects, the coding sequence for the MAD nuclease and the gRNA component sequences are on the same vector. In other aspects, the coding sequence for the MAD nuclease and the gRNA component sequences are on a different vector and in some aspects, the gRNA component sequences are located in an editing cassette which also comprises a donor DNA (e.g., homology arm). In other aspects, the MAD nuclease is delivered to the cells as a peptide or the MAD nuclease and gRNA components are delivered to the cells as a ribonuclease complex.

These aspects and other features and advantages of the invention are described below in more detail.

### BRIEF DESCRIPTION OF THE FIGURES

FIG. 1 is an exemplary workflow for creating and screening mined MAD nucleases or RGNs.

FIG. 2 is a simplified depiction of an in vitro test conducted on candidate enzymes.

FIG. 3 is a simplified depiction of an in vitro nick assay.

FIG. 4 is a cluster map of Type V MADzymes and the structures of various types of Cas12a nucleases.

FIG. 5 shows heat maps of the three active Type V MADzymes identified herein.

FIG. 6 is a table summarizing parameters of the three active Type V MADzymes identified herein.

It should be understood that the drawings are not necessarily to scale.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities. Moreover, all of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, biological emulsion generation, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W. H. Freeman Pub., New York, N.Y.; Cell and Tissue Culture: Laboratory Procedures in Biotechnology (Doyle & Griffiths, eds., John Wiley & Sons 1998), all of which are herein incorporated in their entirety by reference for all purposes. Nuclease-specific techniques can be found in, e.g., Genome Editing and Engineering From TALENs and CRISPRs to Molecular Surgery, Appasani and Church, 2018; and CRISPR: Methods and Protocols, Lindgren and Charpentier, 2015; both of which are herein incorporated in their entirety by reference for all purposes. Basic methods for enzyme engineering may be found in, Enzyme Engineering Methods and Protocols, Samuelson, ed., 2013; Protein Engineering, Kaumaya, ed., (2012); and Kaur and Sharma, “Directed Evolution: An Approach to Engineer Enzymes”, Crit. Rev. Biotechnology, 26:165-69 (2006).

Note that as used herein and in the appended claims, the singular forms “a,” “an,” and “the” include plural referents unless the context clearly dictates otherwise. Thus, for example, reference to “an oligonucleotide” refers to one or more oligonucleotides. Terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible and, in some embodiments—particularly many embodiments in which selection is employed—the transcription of at least one component of the nucleic acid-guided nuclease editing system is under the control of an inducible promoter.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, erythromycin, tetracycline, gentamicin, bleomycin, streptomycin, rhamnose, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The target sequence can be a genomic locus or extrachromosomal locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. As used herein, the phrase “engine vector” comprises a coding sequence for a nuclease to be used in the nucleic acid-guided nuclease systems and methods of the present disclosure. The engine vector may also comprise, in a bacterial system, the λ Red recombineering system or an equivalent thereto. Engine vectors also typically comprise a selectable marker. As used herein the phrase “editing vector” comprises a donor nucleic acid, optionally including an alteration to the target sequence that prevents nuclease binding at a PAM or spacer in the target sequence after editing has taken place, and a coding sequence for a gRNA. The editing vector may also comprise a selectable marker and/or a barcode. In some embodiments, the engine vector and editing vector may be combined; that is, the contents of the engine vector may be found on the editing vector. Further, the engine and editing vectors comprise control sequences operably linked to, e.g., the nuclease coding sequence, recombineering system coding sequences (if present), donor nucleic acid, guide nucleic acid, and selectable marker(s).

### Editing in Nucleic Acid-Guided Nuclease Genome Systems

RNA-guided nucleases (RGNs) have rapidly become the foundational tools for genome engineering of prokaryotes and eukaryotes. Clustered Rapidly Interspaced Short Palindromic Repeats (CRISPR) systems are an adaptive immunity system which protect prokaryotes against mobile genetic elements (MGEs). RGNs are a major part of this defense system because they identify and destroy MGEs. RGNs can be repurposed for genome editing in various organisms by reprogramming the CRISPR RNA (crRNA) that guides the RGN to a specific target DNA. A number of different RGNs have been identified to date for various applications; however, there are various properties that make some RGNs more desirable than others for specific applications. RGNs can be used for creating specific double strand breaks (DSBs), specific nicks of one strand of DNA, or guide another moiety to a specific DNA sequence.

The ability of an RGN to specifically target any genomic sequence is perhaps the most desirable feature of RGNs; however, RGNs can only access their desired target if the target DNA also contains a short motif called PAM (protospacer adjacent motif) that is specific for every RGN. Type V RGNs such as MAD7, AsCas12a and LbCas12a tend to access DNA targets that contain YTTN/TTTN on the 5′ end whereas type II RGNs—such as the MADzymes disclosed herein—target DNA sequences containing a specific short motif on the 3′ end. An example well known in the art for a type II RGN is SpCas9 which requires an NGG on the 3′ end of the target DNA. Type II RGNs, unlike type V RGNS, require a transactivating RNA (tracrRNA) in addition to a crRNA for optimal function. Compared to type V RGNs, the type II RGNs create a double-strand break closer to the PAM sequence, which is highly desirable for precise genome editing applications.

A number of type II RGNs have been discovered so far; however, their use in widespread applications is limited by restrictive PAMs. For example, the PAM of SpCas9 occurs less frequently in AT-rich regions of the genome. New type II RGNs with new and less restrictive PAMs are beneficial for the field. Further, not all type II nucleases are active in multiple organisms. For example, a number of RGNs have been discussed in the scientific literature but only a few have been demonstrated to be active in vitro and fewer still are active in cells, particularly in mammalian cells. The present disclosure identifies multiple type II RGNs that have novel PAMs and are active in mammalian cells.

In performing nucleic acid-guided nuclease editing, the type II RGNs or MADzymes may be delivered to cells to be edited as a polypeptide; alternatively, a polynucleotide sequence encoding the MADzyme are transformed or transfected into the cells to be edited. The polynucleotide sequence encoding the MADzyme may be codon optimized for expression in particular cells, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of the MADzyme to be employed depends on many factors, such as what type of edit is to be made in the target sequence and whether an appropriate PAM is located close to the desired target sequence. The MADzyme may be encoded by a DNA sequence on a vector (e.g., the engine vector) and be under the control of a constitutive or inducible promoter. In some embodiments, the sequence encoding the nuclease is under the control of an inducible promoter, and the inducible promoter may be separate from but the same as an inducible promoter controlling transcription of the guide nucleic acid; that is, a separate inducible promoter may drive the transcription of the nuclease and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

In general, a guide nucleic acid (e.g., gRNA) complexes with a compatible nucleic acid-guided nuclease and can then hybridize with a target sequence, thereby directing the nuclease to the target sequence. With the type II MADzymes described herein, the nucleic acid-guided nuclease editing system uses two separate guide nucleic acid components that combine and function as a guide nucleic acid; that is, a CRISPR RNA (crRNA) and a transactivating CRISPR RNA (tracrRNA). The gRNA may be encoded by a DNA sequence on a polynucleotide molecule such as a plasmid, linear construct, or the coding sequence may reside within an editing cassette and is under the control of a constitutive promoter, or, in some embodiments, an inducible promoter as described below.

A guide nucleic acid comprises a guide polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the components of the guide nucleic acid is provided as a sequence to be expressed from a plasmid or vector and comprises both the guide sequence and the scaffold sequence as a single transcript under the control of a promoter, and in some embodiments, an inducible promoter. In general, to generate an edit in a target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, an intron, a PAM, or “junk” DNA).

The guide nucleic acid may be part of an editing cassette that encodes the donor nucleic acid. Alternatively, the guide nucleic acid may not be part of the editing cassette and instead may be encoded on the engine or editing vector backbone. For example, a sequence coding for a guide nucleic acid can be assembled or inserted into a vector backbone first, followed by insertion of the donor nucleic acid in, e.g., the editing cassette. In other cases, the donor nucleic acid in, e.g., an editing cassette can be inserted or assembled into a vector backbone first, followed by insertion of the sequence coding for the guide nucleic acid. In yet other cases, the sequence encoding the guide nucleic acid and the donor nucleic acid (inserted, for example, in an editing cassette) are simultaneously but separately inserted or assembled into a vector. In yet other embodiments, the sequence encoding the guide nucleic acid and the sequence encoding the donor nucleic acid are both included in the editing cassette.

The target sequence is associated with a PAM, which is a short nucleotide sequence recognized by the gRNA/nuclease complex. The precise PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve fidelity, or decrease fidelity. In certain embodiments, the genome editing of a target sequence both introduces a desired DNA change to a target sequence, e.g., the genomic DNA of a cell, and removes, mutates, or renders inactive a proto-spacer mutation (PAM) region in the target sequence. Rendering the PAM at the target sequence inactive precludes additional editing of the cell genome at that target sequence, e.g., upon subsequent exposure to a nucleic acid-guided nuclease complexed with a synthetic guide nucleic acid in later rounds of editing. Thus, cells having the desired target sequence edit and an altered PAM can be selected using a nucleic acid-guided nuclease complexed with a synthetic guide nucleic acid complementary to the target sequence. Cells that did not undergo the first editing event will be cut rendering a double-stranded DNA break, and thus will not continue to be viable. The cells containing the desired target sequence edit and PAM alteration will not be cut, as these edited cells no longer contain the necessary PAM site and will continue to grow and propagate.

As mentioned previously, the range of target sequences that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs). Because the mined MAD nucleases disclosed herein may recognize different PAMs, the mined MAD nucleases increase the number of target sequences that can be targeted for editing; that is, mined MAD nucleases decrease the regions of “PAM deserts” in the genome. Thus, the mined MAD nucleases expand the scope of target sequences that may be edited by increasing the number (variety) of PAM sequences recognized. Moreover, cocktails of mined MAD nucleases may be delivered to cells such that target sequences adjacent to several different PAMs may be edited in a single editing run.

Another component of the nucleic acid-guided nuclease system is the donor nucleic acid. In some embodiments, the donor nucleic acid is on the same polynucleotide (e.g., editing vector or editing cassette) as the guide nucleic acid and may be (but not necessarily) under the control of the same promoter as the guide nucleic acid (e.g., a single promoter driving the transcription of both the guide nucleic acid and the donor nucleic acid). For cassettes of this type, see U.S. Pat. Nos. 10,240,167; 10,266,849; 9,982,278; 10,351,877; 10,364,442; 10,435,715; and 10,465,207. The donor nucleic acid is designed to serve as a template for homologous recombination with a target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the target sequence (e.g., a homology arm). When optimally aligned, the donor nucleic acid overlaps with (is complementary to) the target sequence by, e.g., about 20, 25, 30, 35, 40, 50, 60, 70, 80, 90 or more nucleotides. In many embodiments, the donor nucleic acid comprises two homology arms (regions complementary to the target sequence) flanking the mutation or difference between the donor nucleic acid and the target template. The donor nucleic acid comprises at least one mutation or alteration compared to the target sequence, such as an insertion, deletion, modification, or any combination thereof compared to the target sequence.

Often the donor nucleic acid is provided as an editing cassette, which is inserted into a vector backbone where the vector backbone may comprise a promoter driving transcription of the gRNA and the coding sequence of the gRNA, or the vector backbone may comprise a promoter driving the transcription of the gRNA but not the gRNA itself. Moreover, there may be more than one, e.g., two, three, four, or more guide nucleic acid/donor nucleic acid cassettes inserted into an engine vector, where each guide nucleic acid is under the control of separate different promoters, separate like promoters, or where all guide nucleic acid/donor nucleic acid pairs are under the control of a single promoter. In some embodiments the promoter driving transcription of the gRNA and the donor nucleic acid (or driving more than one gRNA/donor nucleic acid pair) is an inducible promoter. Inducible editing is advantageous in that isolated cells can be grown for several to many cell doublings to establish colonies before editing is initiated, which increases the likelihood that cells with edits will survive, as the double-strand cuts caused by active editing are largely toxic to the cells. This toxicity results both in cell death in the edited colonies, as well as a lag in growth for the edited cells that do survive but must repair and recover following editing. However, once the edited cells have a chance to recover, the size of the colonies of the edited cells will eventually catch up to the size of the colonies of unedited cells. See, e.g., U.S. Pat. Nos. 10,533,152; 10,550,363; 10,532,324; 10,550,363; 10,633,626; 10,633,627; 10,647,958; 10,760,043; 10,723,995; 10,801,008; and 10,851,339. Further, a guide nucleic acid may be efficacious directing the edit of more than one donor nucleic acid in an editing cassette; e.g., if the desired edits are close to one another in a target sequence.

In addition to the donor nucleic acid, an editing cassette may comprise one or more primer sites. The primer sites can be used to amplify the editing cassette by using oligonucleotide primers; for example, if the primer sites flank one or more of the other components of the editing cassette.

In addition, the editing cassette may comprise a barcode. A barcode is a unique DNA sequence that corresponds to the donor DNA sequence such that the barcode can identify the edit made to the corresponding target sequence. The barcode typically comprises four or more nucleotides. In some embodiments, the editing cassettes comprise a collection of donor nucleic acids representing, e.g., gene-wide or genome-wide libraries of donor nucleic acids. The library of editing cassettes is cloned into vector backbones where, e.g., each different donor nucleic acid is associated with a different barcode.

Additionally, in some embodiments, an expression vector or cassette encoding components of the nucleic acid-guided nuclease system further encodes one or more nuclear localization sequences (NLSs), such as about or more than about 1, 2, 3, 4, 5, 6, 7, 8, 9, 10, or more NLSs. In some embodiments, the nuclease comprises NLSs at or near the amino-terminus of the MADzyme, NLSs at or near the carboxy-terminus of the MADzyme, or a combination.

The engine and editing vectors comprise control sequences operably linked to the component sequences to be transcribed. As stated above, the promoters driving transcription of one or more components of the mined MAD nuclease editing system may be inducible, and an inducible system is likely employed if selection is to be performed. A number of gene regulation control systems have been developed for the controlled expression of genes in plant, microbe, and animal cells, including mammalian cells, including the pL promoter (induced by heat inactivation of the CI857 repressor), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, CA); Bujard and Gossen, PNAS, 89 (12): 5547-5551 (1992)), the Lac Switch Inducible system (Wyborski et al., Environ Mol Mutagen, 28 (4): 447-58 (1996); DuCoeur et al., Strategies 5 (3): 70-72 (1992); U.S. Pat. No. 4,833,080), the ecdysone-inducible gene expression system (No et al., PNAS, 93 (8): 3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

Typically, performing genome editing in live cells entails transforming cells with the components necessary to perform nucleic acid-guided nuclease editing. For example, the cells may be transformed simultaneously with separate engine and editing vectors; the cells may already be expressing the mined MAD nuclease (e.g., the cells may have already been transformed with an engine vector or the coding sequence for the mined MAD nuclease may be stably integrated into the cellular genome) such that only the editing vector needs to be transformed into the cells; or the cells may be transformed with a single vector comprising all components required to perform nucleic acid-guided nuclease genome editing.

A variety of delivery systems can be used to introduce (e.g., transform or transfect) nucleic acid-guided nuclease editing system components into a host cell. These delivery systems include the use of yeast systems, lipofection systems, microinjection systems, biolistic systems, virosomes, liposomes, immunoliposomes, polycations, lipid: nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in, e.g., U.S. Pat. Nos. 10,435,713; 10,443,074; 10,323,258; and 10,415,058.

After the cells are transformed with the components necessary to perform nucleic acid-guided nuclease editing, the cells are cultured under conditions that promote editing. For example, if constitutive promoters are used to drive transcription of the mined MAD nucleases and/or gRNA, the transformed cells need only be cultured in a typical culture medium under typical conditions (e.g., temperature, CO₂ atmosphere, etc.) Alternatively, if editing is inducible—by, e.g., activating inducible promoters that control transcription of one or more of the components needed for nucleic acid-guided nuclease editing, such as, e.g., transcription of the gRNA, donor DNA, nuclease, or, in the case of bacteria, a recombineering system—the cells are subjected to inducing conditions.

### Examples

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1: Exemplary Workflow Overview

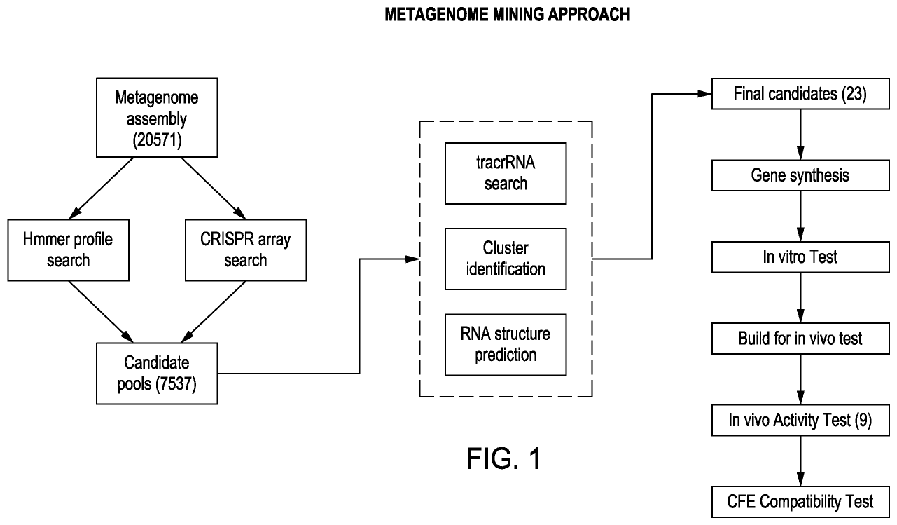

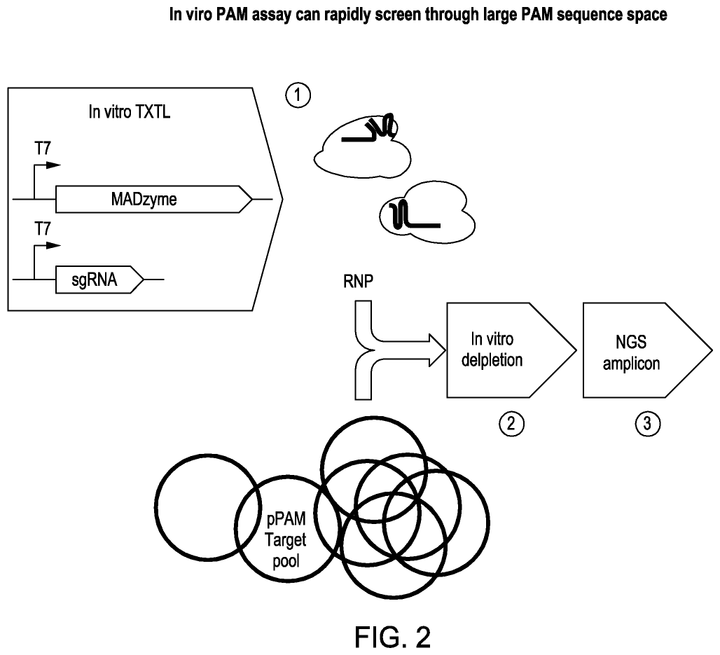

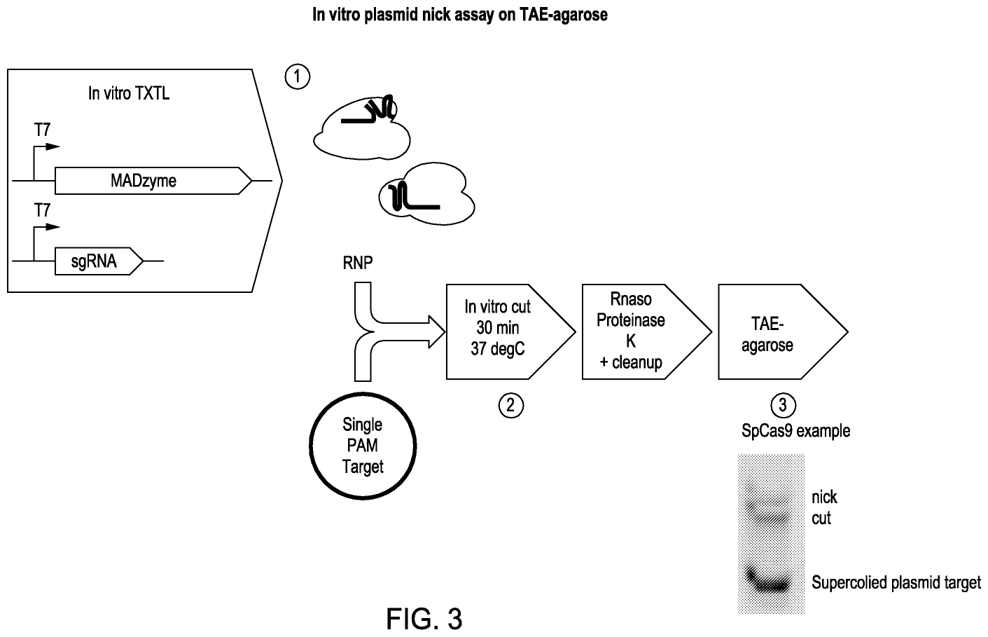

The disclosed MADzyme Type V CRISPR enzymes were identified by the method depicted in FIG. 1. FIG. 1 shows an exemplary workflow for creating and for in vitro screening of MADzymes, including those in untapped clusters. In a first step, metagenome mining was performed to identify putative RGNs of interest based on, e.g., sequence (HMMER profile) and a search for CRISPR arrays. Once putative RGNs of interest were identified in silico, candidate pools were created and each MADzyme was identified by cluster, the tracrRNA was identified, and the sgRNA structure was predicted. Final candidates were identified, then the genes were synthesized. An in vitro depletion test was performed (see FIG. 2), where a synthetic target library was constructed in which to test target depletion for each of the candidate MADzymes. After target depletion, amplicons were produced for analysis by next generation sequencing. FIG. 2 depicts the in vitro depletion test in more detail. FIG. 3 depicts the in vitro plasmid nick assay performed.

### Example 2: Metagenome Mining

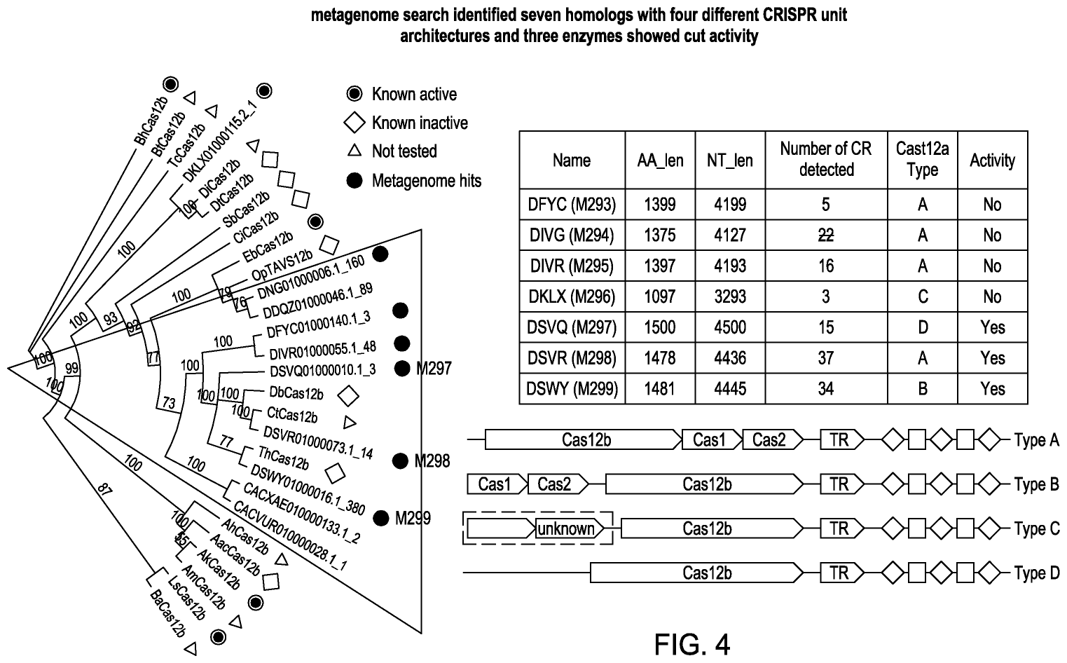

The NCBI Metagenome database was used to search for novel, putative CRISPR nucleases using HMMER hidden Markov model searches. Hundreds of potential nucleases were identified. For each potential nuclease candidate, putative CRISPR arrays were identified and CRISPR repeat and anitirepeats were identified. Seven nucleases were chosen for in vitro validation and three active MADzymes were identified. The Type V cluster is shown in FIG. 4. MAD297, MAD298 and MAD299 were found to be active. The table in FIG. 4 gives some parameters for the seven nucleases chosen, and the structures of the Cas12a types (A, B, C and D) are shown below the table. Table 1 below lists the identified MADzymes, including amino acid sequences and the nucleic acid sequences of the CRISPR RNA and the trans-activating crispr RNA.

TABLE 1

| MAD | | | | |
|---|---|---|---|---|
| name | Contig_id | aa_seq | CRISPR repeat | tracrRNA |
| MAD293 | DFYC | MAAFQRSYTMNLKPATSEQDKFILWNRLFLTHWSVNEGAK | GTTTAAGTAGA | GGGAAGGGCC |
| | 01000140.1 | IFGELFLNLRGGLSPELDIFDLDKVKDDKKKKAFVMGRRRLLA | TACTACTGAAA | TATTTCCCAGC |
| | | LGWLSVEDNLSAGEHPFRIREIPVGRNMGKSQASTLLTEILK | AGACCGATGG | ATGTGTCTTCG |
| | | NKGIKDEAVIKEWIDDCTPSLIANIREDAVWINRASSFNSITP | ACACA | CATTTAATTGC |
| | | CPTKDEVWIVLSGLLGLRFLDLSLEEVKGKETEYLYDLGEKET | [SEQ ID NO: 2] | TTTAGCACTGG |
| | | QSKSDPSKKARELFGNLFTQNPVLMKNSRDKKDTFAKEFYL | | GCATCGTTCTT |
| | | AFKEFKDYEKLKEKIESWRKEKEFPLIENPVAEKYPPEVTFTGS | | TTTAGTAGT |
| | | PCTVSKRYRKLLVSLELWPSSQDENGNIPKTEKTEDKTHNQV | | [SEQ ID NO: 3] |
| | | LLDYLLKACNEGNKGTQKIITPVWANNLKAELELKMNEIIRIG | | |
| | | ESSSTELQRLMIKMAARRISQTLSWIKINEQTKHDAYQKKNK | | |
| | | AFKLLSEIDKNGEACKWLENYELFRTDDSGGEEYHISLRAISC | | |
| | | WKQILEEWQKNDSPKALREKVKEVQAEEEKFGDARLFEDLA | | |
| | | DDNARSVWLLPDGNKTPDILNWWCEYRTAEIDESRFKIPCY | | |
| | | ICHPHPFKHPVYVEYGKSNPKVIFAMKNNKVKKGHIEHGWN | | |
| | | PQNPRSIALSLFNNGNRESSLVPFIWESKRLWKDLGGEATQI | | |
| | | GDIPRSDRMGLSGKRESVKPKAPFQKEVWNARLQSDRRTLE | | |
| | | KLEKYWNPESMKWIDDGKFLIQSKWFITFGPDMETAEGPW | | |
| | | KLYLKEKYVDNKILGNRSKENQKRGYRAKKLLSGYPAGMRIL | | |
| | | SVDLGHRYAASCAVWETITKKQITEELAYQPDNNSLFEHSCK | | |
| | | TIDKKIKNTVYRRIGEDSIDAPWAKLEKQFTIKLQGEDKSCYLL | | |
| | | RSDEKELFRSILSKLSCLNNDTGHNILEMIENLLRIVKAKIYRQ | | |
| | | GILARISYSMTAQYKPGKGGQKSPLSDEDKIHYLSENLAAWS | | |
| | | AIMGNQEWNEDVISDWYKTYISHLVSGPKPKEGNRKSDRD | | |
| | | KIIEYFLPAARKLYDDNETRIKIHDLFKELWDENNKQLSAVLKE | | |
| | | IKKIILPKGIRYFDKNTDNPSKWKNNQSKLKQITHRGGLSMQ | | |
| | | RIVAIEEYYKLAKAYKNHPEPDDLTKNIPLPGDNSSAGFNQRI | | |
| | | RDTLERMKEQRVKQIASRIVESALGLGIEGYKKRPLTPENKPC | | |
| | | QAIVIEDLSHYRPDELQTRRENRRLMQWSSSKVKKYLKEACE | | |
| | | MHDVRLVEISPEYTSRQDSRTGAAGLRCIDINIREFLKDSSR | | |
| | | WQNKINTIQKKPANKKSNLDQYLIELNESLGNKYKDKVIPSD | | |
| | | NFVRIPRKGGDVFVSSSKESPVSKGIQADLNAAANIGLKALL | | |
| | | DPDWAGAWWYILIEVKSNHVIPYGEKYKGSECLRAWKFSGL | | |
| | | ENQVMKNNMNLWRDLQSQFSGEDKWMSYKEYNELTEKR | | |
| | | VINILRERAGLELIKE | | |
| | | [SEQ ID NO: 1] | | |
| MAD294 | DIVG | MNRIYQGRVSKIEIKDSEGNFRNVPVGSPDTCPLWRHHRIF | GCCGCAGCCCC | GCCCCGATTTC |
| | 01000006.1 | QDAVNYYLVALGALAGTGSENAFVGLGSKDRVIHDLYSRLF | CGCGATGGGA | CCTTTGAATGA |
| | | DSWERFPRDMHGASSLRDSLRRTLPGLSERASLQDAFDAILS | AAGAGATTGT | TCTCGGCCTCG |
| | | GNEANARERVLSLLSLIQDLGGDIQKGSKRYFPFFCEPATKAT | GGCGG | TTGCCACTGAC |
| | | FPRARVGLLKVEGKDFVPRLLWSSDLEIAPDQVVEQLKFEYF | [SEQ ID NO: 5] | CGAATTCTTCC |
| | | ANPNESVQPIEGNEARVRLIEALDNPQLGIELPIEILSDLRKRV | | GCCTTTGGAAT |
| | | HLIETDIRIPRYFFGGAGAELRKFRLDLFLIAAYVTPDPSILRAL | | TCCAAGCTCTT |
| | | RNSFKEPSASKSSKKKDETEEVENLLRSLGDDPLILARGERGF | | TGACATCGCGA |
| | | VFPSFTSLPTWVGANAQKPIWRDFDIAAFAEALKSLNQFTA | | GCGCTGAGG |
| | | KTEEREEKLKKAEETLHYMLGISDAIPRSSDSETEEQAPSRPG | | [SEQ ID NO: 6] |
| | | KDPRWPLVAQLEKELGENLSEGTWQLSRSAMRGLRDIIGL | | |
| | | WRKHPGASVVTLQKDVKTYQADEKHKREIGSVOLFLLLCEE | | |
| | | RYHALWQTETDDERGDESEENDDPARILSDAIEVHQIRREVE | | |
| | | RFREPIRLTPAEPVFSRRLFMFSDLTDKLAKVKFGETTEENSE | | |
| | | VKSQFVEAAIALKEGENLKEARVRITFSAPRLHRDELLGGAES | | |
| | | RWLQPITAALGFSNPAPSVKFDSAVALMPDHMDDGRIRHL | | |
| | | LNFPVNFDSAWLHQSIGKADLWKSQFNGTKDKNLHLHWA | | |
| | | GTARDTTRKNTWWENRTIIENGFTVLSNDLGQRSAGAWAL | | |
| | | LKVTCSRPDTKHPVRSIGHDGTREWFATVLATGIHRLPGED | | |
| | | QRILKNGKWATEQSGKKGRNATFSEYEAACVLAKNLGCESV | | |
| | | ENWLGMSGEKSYPALNDQLVKIANRRITRLGTYHRWSCFSP | | |
| | | EKFEDPARRANVIGGQLAELSAYQDENVTVSADILKSGDFEG | | |
| | | FRHRAGAAFEALRTELEVHLVNLANLTAPLRQKVWSWQKR | | |
| | | PDSSGYGDLLMVDLDDCHPKIRGQRGLSMARLEQLEGLRRL | | |
| | | FLRYNRSLDRSPGIPAKFGREDVGRTSGEPCQALLVKIDRMK | | |
| | | EQRVNQTAHLILAQALGVRLCPHRIEENERKSRDLHGEYEKI | | |
| | | PGREPVDFIVIEDLSRYLSSQGRAPSENSRLMKWAHRAVRD | | |
| | | KLKMLAEEPFGIPVVETVPAYSSRFHALNGQAGSRLHELHEL | | |
| | | EAYQQQSLINLAAKTDFQNRDRSKAAGELFEQFQALAKLNE | | |
| | | RRRAEGKKVPRTLYYPKSGGPLFLASRDGDTIHADVNAAINL | | |
| | | GLRAIAAPACIDIHRRLRATKEKEVYRPRVGNAREKSAFSKDD | | |
| | | IIQPSGAPSKKFASSSSPNFFYEPEDLKQANGEPLFDRAMFG | | |
| | | EYSLVSGVSLWSMVNNAIYIRCVELNRTRLHGKDPDDQIPM | | |
| | | [SEQ ID NO: 4] | | |
| MAD295 | DIVR | MAAFQRSYTMNLKPATSEQDKFILWNRLFLTHWSVNEGAK | GTTTAAGTAGA | GGGAAGGGCC |
| | 01000055.1 | IFGELFLNLRGGLSPELGIFDLNKDKDDRKKKALVMGRRRLLA | TACTACTAAAA | TATTTCCCAGC |
| | | LGWLSVEDNLSAGDHPFRIREIPVGRNMEIKQATTLLTEILKN | AGACCGATGG | ATGTGTCTTCG |
| | | KGVKDEAVIKEWIDDCTPSLIANIREDAVWINRAKSFYSMNP | ACAC | CATTTAATTGC |
| | | CPTKDEVWKILSYVLNTSFLDLSLNDSSERDNTKNKKGTKEN | [SEQ ID NO: 8] | TTTAGCACTGG |
| | | EKDVSNKSKELYGWLFTKNPNKMREAGENKDKFINNFRENF | | GCATCGTTCTT |
| | | NTFTDYANLKVEIELWRKNNISNTLLITQKAKYPPEVKEANH | | TTTAGTAGT |
| | | PSKFSVGYRKLLVHLELWPSSKDENGDIPKGIEGKDKSHNQIL | | [SEQ ID NO: 9] |
| | | LDYLLEVCNEGNKTTKKVIVPAWADGIKTELESKASIKVGDST | | |
| | | SSVLQRLMIKMAARRISQTLSWIKINEQVRHDAYQKKNKAF | | |
| | | KLLCEIDKNGEACKWLENYELFRRDDSGGEEYHISARAISCW | | |
| | | KQILEEWQKNDSSKALREKVKVVQAAEDKFGDARLFEDLAD | | |
| | | DNARSVWLLPDGNKTPDILNWWCEYRTADIDESRFKIPCYC | | |
| | | HPHPFKHPVYVEYGKSNPQVIFSLKHDKARKNRIDNGWNPK | | |
| | | NPRILALLLLDIVRQKSTLAPFVWESKRLWKDLGGDATVTYKI | | |
| | | PRSDRMGLSSIGNIDYARPEVPFLKEKWNARLQSDRRTLEKL | | |
| | | EKYWNPESMKWIDDGKFLIQSKWFITFGPDMETAEGPWKL | | |
| | | YLKENINDNNYLGNRSKENQKRGYRAKKLLSGYPAGMRILS | | |
| | | VDLGHRYAASCAIWETITKKQITEELAYQPDKNSVFEHSCKTI | | |
| | | DKKIKNTVYRRIGDDSIDAPWAKLEKQFTIKLQGEDKSCYLLR | | |
| | | SDEKELFRSILSKLSCLNNDTGHNILEMIENLLRIVKAKIYRQGI | | |
| | | LARISYSMTAQYKPGKGGQKSPLSDEDKIHYLSENLAAWSAL | | |
| | | MGNQEWNEDGISDWYKKYISHLVSGPKPKEGNRKSDRDKII | | |
| | | EYFLPAARKLYDDNETRINIHDLFKELWDENNKQLSAVLKEIK | | |
| | | KIILPKGIRYFDKNNDSSSRWKNNQSKLKQITHRGGLSLRRIV | | |
| | | AIEGYYKLAKAYKNHPEPDNLTKNIPLPGDNSSAGFNQRIRD | | |
| | | TLERMKEQRVKQIASRIVESALGLGIEGYKKRPLTPESKPCQA | | |
| | | IVIEDLSHYRPDELQTRRENRRLMQWSSSKVKKYLSEACEM | | |
| | | HDVLLVEISPEYTSRQDSRTGVAGLRCIDINIREFLKDSSSWQ | | |
| | | NKIKTIQMKPTNKKSNLDQYLIELNESLGERYKDKVIPSDKFV | | |
| | | RIPRKGGDIFVSSSKESPVSKGIQADLNAAANIGLKALLDPD | | |
| | | WAGAWWYILIEAKSNHVIPYGKKYKGAECLRDFKFSGLENQ | | |
| | | VMKNNMNLWRDLQSQFSSEDKWMSYKEYNELTEKRVINIL | | |
| | | RERAGLELIEE | | |
| | | [SEQ ID NO: 7] | | |
| MAD296 | DKLX | MVTRALNLKLVVPRRPGELTKAEALWSTHDIVNRATSYYES | GGTGAAGTCA | ATGTAACCCTA |
| | 01000115.1 | QLLLCRQQDYQTRELTVSAGDQAPDLDALIANARDRNRYRG | CCCCCGTTTTG | TAGGGGTTGC |
| | | LEKPQVVREKLRNLYEAIVPPAIGKTGTAQAVGAFVSPLLDA | TAGGCCTACTG | GTGAGTCGGC |
| | | DSRGFTEIFDKIEALPNWVDGVRAEEPDALEAAADWLKSPQ | GCAC | CATAGTGCCTC |
| | | GKERLRPTGAPPTWIKLAKKKDAGWAAAFVADIDKKLKEVE | [SEQ ID NO: 11] | GGCAACAGCG |
| | | GTPTLMQELRALGVMPLFPSFFASRIAGHKGAVSTWDRLAL | | TAAAAAACTGC |
| | | RLAVAHLLSWESWVELAAKEHAARVAKLEKFRDDNILGEIA | | TGCCAGTGGTC |
| | | DAVEALRLYEKERTEELQQKAQLDAEEVRTTSRTIRGWVDLR | | GAAGTAAGTC |
| | | EKWLKTDASPDALISLVAAEQKRKSGKFGDPQLFRWLAKPE | | AACAAAACGG |
| | | NHFVWNKPDFDPPSLFASLRMIEGLVERSKETAWMTLPDA | | AGGT |
| | | RLHPRSSQWEPHGGGNLKTFRLEQGEGGSLSVTLPLLRKSG | | [SEQ ID NO: 12] |
| | | DDSYVEEEHAFSLAGSKQIPNASLDVRRNKYCLSYRTPTGEE | | |
| | | AEAVVGSADLLLDWYFLQQRSEHRPEEGDIGPAFLKLALDIT | | |
| | | PIDPVWGEREKTPAIHHFKTASGKNTRHADGVAPGFRMLA | | |
| | | VDLGIRTLATCSVFELKATAPAGRLSFPIAHLDLHAVHERSFTL | | |
| | | TLDGEDPDRDAERWRENKSAELRRLRMGLTRYRNIRNMRE | | |
| | | DAPDEREVLLEDLQEKVQEHGWAFEEPLLRELAKHKDTPEPI | | |
| | | WEAELTKALAQFRSDFGVIVGEWRRSNRARSTDSHAGKSM | | |
| | | WAIDHLTNSRRFLMSWSLLSKPGQIRRLDRDKQGVFAKHLL | | |
| | | DHLEGLKADRLKTGSDLIVQAARGFRRDKRGNWHKAYKPC | | |
| | | HGILFEDLSRYRMRTDRPRRENSQLMKWAHRAVPKEVGM | | |
| | | QAEVYGIRVEDTGAAFSSRFHAASHTPGIRMHPICQKDLEN | | |
| | | EWLLDEIEKQNSGVKRRELKLGQLVQLNGGELFACVTASGV | | |
| | | KTLHADINAAQNLQRRFFTRHGDAFRIVARKVLVDEEEVWV | | |
| | | PRSLGKRLLGALGSHGKLVPTGHESGSCRFEEITTRAWSKLS | | |
| | | GEKLSDDRVGNEEDQIIASIEEEALERTGEVVVFFRDPSGQVL | | |
| | | PRDLWYPSKTFWSIVKSTTLSKLKAAP | | |
| | | [SEQ ID NO: 10] | | |
| MAD297 | DSVQ | MATAINYPTTQRAYTLRLRGIDPQDQSWRDALWATHEAVN | GGCGGCGAAT | CGGAGGGAAC |
| | 01000010.1 | RGAKVFGEWLLTLRGGLDHQLADAPVKVRGGTTRLPSDEER | TCGCCGGCATC | TCCGTGAACGT |
| | | RDRRVLLALSWLSVEDAHGAPPDASLIVAKGTDSADCRARK | TCGATCGACCG | GTCTTCCCCTT |
| | | LADALIAILQARSVAASEIGDPSKPPEDQPGTWLGDCMGSLS | ACAC | CGATGGGCTT |
| | | AAIRDDAVWVNRSKAFDAATQSCPSLTRDEIWDFLEPFFAS | [SEQ ID NO: 14] | GGCACACGGG |
| | | PDAYLKPERAESDEGDSTSAATEDKAKDLVQKAGGWLSKR | | GTCAATCGAGT |
| | | MGAGGGANFQDLARAYQAIAQWASSAQPGQSAQQAVGS | | TGCCGTTGAA |
| | | LAGYLSQHGFSPTANDATGVLAVISGPGYKSATRNHITAIATS | | [SEQ ID NO: 15] |
| | | PEITPQDLSKLQELATKDKAGCSSKIGGKGPRPYATMILQQV | | |
| | | EAACGFTYLQSDGPARHREFSVMLDHAARRVNVAHSWIKN | | |
| | | AEAERRQFESDARRIKKVPQDALNWLRGYCEERGGASGSLE | | |
| | | GYRIRRRAIDGWDQVVIRWSRSDCQSADDRIAAARQLQDD | | |
| | | PEIDKFGDIQLFEALAAEEALCVWKPDGNPTAQPLKDFVAAT | | |
| | | EADAKKKRFKVPAYRHPDPLRHPVFTDFGNSRWGIEYSAHR | | |
| | | APAKCDELGQQVDRLTQAVAEAQRNLDGATAAQRASRESK | | |
| | | LAEAQSKLVAAQTEFAAINDPHRVELKLWNGQAVAAIPMR | | |
| | | WSSKRLIADLSLRRATQPSSDQRIGVTRADRLGRAAGNADD | | |
| | | GRPVTITGLFQQDHWNGRLQAPRAQLDAIAKHVDKHGWD | | |
| | | AKARRQIARIRWVVSFSAELSQQGPWFEFCHRFGEDAPARP | | |
| | | FVSRHGEYAVKHRDNDQRKGHAKLILSRLPGLRVLAVDLGH | | |
| | | RYAAACAVWEAISSDQMRQACAAANAPAPHPLAMYIHLKS | | |
| | | TTAKGKPTTTIYRRIGPDKLPDGTPHPAPWARLDRQFLIKLP | | |
| | | GEDRPARAASPDEIKAVEDFEDSVGRVRTAVDPPRKRGVDL | | |
| | | LMHDAVRTARLALARHGRRARIAFQLISQVRILPGGRPQTLD | | |
| | | DAGRRDLLNDTLADWYALATDSRWTDAAARQLWNERLAA | | |
| | | LNGGFTIDPPADASQPEAERTRAQRRQAEQELRHRLASLVE | | |
| | | ALFCNPTLCQQLHQAWTDRWNADDQQWRSRLKWLSRW | | |
| | | LLPRGGSRRDGSRRHVGGLSLTRISTLIDFRRKVQVGYFTRLR | | |
| | | PDGSRAEIGPQFGQSTLDAIQRLKDQRIKQLTSRIVEAALGIG | | |
| | | VEQDRIWDAAKRKWRTVKRPREPRYHVDDQGVQQRDPRF | | |
| | | QACHAVVIEDLSHYRPEETRTRRENRATMDWKSAETRKRLA | | |
| | | DHCQLYGLHLRDVNPQYTSRQDSRTGAPGCRCVDVSVADF | | |
| | | LTKPAWRKQVAQARGKVASNRGDARDRLLVELDHQLTTAN | | |
| | | SLRGEMDSLRIPVNGGEVFVSADPRSPLAAGIQADLNAAAN | | |
| | | IGLRALMDPDFLGTWWYVPCDPSTKKPHIEKVKGSILATVG | | |
| | | ALQATSEEAAAPPRRGRGGTRSAAPREVINLWRDPSAVRIQ | | |
| | | DATAGEVWDVTPVYWSIVKDRVVDVLRQRNTKSGD | | |
| | | [SEQ ID NO: 13] | | |
| MAD298 | DSVR | MSQQVKPPVTQRAYTLRLRGIDPSDTSWRKALWQTHEGV | GTTGCCGACGT | TTGCCTCTACA |
| | 01000073.1 | NKGAKAFGDWLLTLRGGLDHTLADTKVKGGKGKPDRDPTD | CAGCACCAACC | GGAGGCGAGA |
| | | EERKARRILLALSWLSVESKLGAPVGFIIASGTEAAEDRNRKV | TGATCGACGG | ATGCCACGGCA |
| | | VAALEEILKSRNVATNEIDQWKNDCSASLSAAIRDDAVWVN | ACAC | CGTGTCTTCCC |
| | | RSKAFDEAVKSIGSSLTREEAWDMLERFFGSRDAYLAPAKGS | [SEQ ID NO: | CTTCAATGGGC |
| | | EDESSETEQEDKAKDLVQKAGQWLSSRFGTGKGADFCRMA | 17] | TTGGCACCGTG |
| | | NVYGKIAAWADNAQADTTGNDAINNLAAALNEYSPEPNDL | | GAGTCGATCA |
| | | KGVLGLISGPGYKSATRNLLNQLAAKATVTQQDFVSLKDKAS | | GTTTTGTGCCG |
| | | NDAQKCKQNTGSKGPRPYSDAILKNVESVCGFTYLQDGGPA | | GCGAAGG |
| | | RHSEFAVILDHAARRVSLAHTWIKRAEAERRKFEEDAKKIDQ | | [SEQ ID NO: |
| | | VPKAAKDWLDSFCLERSGASGALEPYRIRRRAVDGWKEVVA | | 18] |
| | | AWSKADCKTAEDRIAAARALQDDPEIDKFGDIQLFEALAED | | |
| | | DAVCVWHKDGDAAKPPDPQPLIDYALAAEAEFKKRHFKVP | | |
| | | AYRHPDALLHPVFCDFGNSRWDICFDVHKNVQTPFPRALSL | | |
| | | TLWTGSGMVSVPLCWQSKRLARDLALGQNAQNDGSSEVT | | |
| | | RADRLGRAASNVTKSDEVKISGLFEQEDWNGRLQAPRQQL | | |
| | | EAIAAVRDNLSLSDQERERRMSGMIDRIRWLVTFSAKLQPQ | | |
| | | GPWCEFAEKIQIGINPQYWPHADTNKDRKGHARLILSRLPG | | |
| | | LRVLSVDLGHRYAAACAVWEAVNAEQINVACRAAGHREPK | | |
| | | ASDLYLHLKRKTTKQKKGDQVEFKETTIYRRIGADTLPDGTPH | | |
| | | PAPWARLDRQFLIKLQGEEEGVRAASNEEIWAVHRLEVELG | | |
| | | RTAPLIDRLVKAGWGQSGKQKTRLDALRNLGWAPANEVQ | | |
| | | GSDEMDEGEVHKPSLSVDELMSSAVRTMRLALKRHGDRAR | | |
| | | IAFAMTAAYKPMPGDRKYYFTEAKDASANEDATARNGKHIE | | |
| | | FIQDALLLWYGLTSSRGWRDDAARQLWDDHIAKLSVYKAPE | | |
| | | EIGEDASGVERKTKQKENREKLHDVAKALAQDVNLRKVLHN | | |
| | | AWKKRWEKDDERWKKQLRWFKDWVFPRGKHASDPAIRK | | |
| | | VGGLSLPRLATLIEFRRKVQVGFFTRLQPDGTRAETKEQFGQ | | |
| | | SALDTLEHLREQRVKQLASRIVEAALGIGRVRRPLGGKDPKR | | |
| | | PDVRVDEPCHAIVIEDLTHYRPEETRTRRENRQLMTWSSSK | | |
| | | VKDYLSEACQLHGLHLREVSASYTSRQDSRTGSPGIRCQDVP | | |
| | | VKEFMRSPFWRKQVAQAEKKGDAHERFLCELNAMWKDKT | | |
| | | VADWEKAGAVRVPLKGGEVFVSADRISPSAKGLQADLNAA | | |
| | | ANIGLRALTDPDWPGKWWYVPCEPASFRPAKDKVDGSAV | | |
| | | VNPGQPLRQSAQAQSGDAAKDKKKRGNKGAGQSKEVVNL | | |
| | | WRDISSSPLECIECGEWKEYAAYQNEVQYRVIRILEEQIKGRD | | |
| | | RQPHEGSREDDIPF | | |
| | | [SEQ ID NO: 16] | | |
| MAD299 | DSWY | MARRSASTSKPPGPVAPTTQRAYTLRLRRAPGKCPHCEQDA | GCCGCGTCGG | GTCGCCTATAG |
| | 01000016.1 | CDCWREALWATHAAFNRGAKAFGDWLLTLRGGLSHELAE | CCGACGCGGC | GGCGATACAA |
| | | QPSPPKNKEPTCEESDAIRKNRRILLALSWLSVEDDCSAPTGT | CTTGATCGATG | CTCCGAGCATG |
| | | FRVASGKDSEAERKNKVLTAFRSILTARRMRSQDVESWIAD | GACAC | TGTCTTCCCCTT |
| | | CAASLSAKIREDAVWINRSACFDQRALDLKVLSREYAKAAV | [SEQ ID NO: | CAATGGGCTTG |
| | | MSFFGPLDEYFKLPDEADDTKPAVGGDGPDFRTLARQWVS | 20] | GCACTCGGCAT |
| | | TNFGTGKKSDSEAIAQNLRKLADANLAPFSGKPKAALIAHLS | | CGATCAAGCTC |
| | | VELDGSTADIDGLCRAIGWNTGRPSKGRVAIERLPDPPTETSI | | GCGTCGGCTGT |
| | | QTMQQKFREEAEAKASSKGLRQVPEWMPAFQKSIERDCG | | CGGGCC |
| | | MPFKLGEGRDHIGEFSVMLDHAARRVSIGHSWIKRAEAERR | | [SEQ ID NO: |
| | | RFEADAQRLNHIPAAAKDWLDQFVQFRSGSSGAAAAGGEY | | 21] |
| | | RIRRRAIEGWDEIIKRWKRAACKSPEDRVAAAREVQADPEIE | | |
| | | KFGDIQLFEALAADDAECVWRGDGNGTPDPLKDYVAATDA | | |
| | | LDKMRRFKVPAYRHPDPLAHPVFGDFGNSRGDIRFAVHEA | | |
| | | AKATRGTKRIAKDQKEWIRERHGLRMGLWDGQSVRTADLR | | |
| | | WSSKRLVDDLALRNHVTTRRTGPVSRADRLGRAAAGLGAD | | |
| | | EAACVAGLFELPDWNGRLQAPRAQLDAIAACVAANGGKW | | |
| | | DDKARKLRDRIEWLVSFSAKLECCGPFMEYASQNGIQPNGK | | |
| | | GEYYPHAERNKGRTGHAKLILSRLPGLRVLAVDLGHRFAAAC | | |
| | | AVWEALSKIAFDAETKGREVVSGGRAADDLYCHTRHLDCAG | | |
| | | KARTTIYRRIGPDKLPDGSDHPAPWARLDRQFLIKLOGEERP | | |
| | | ARAAGPAETAAVQQIETDLGRARGQEDLPPRPVDSLMREA | | |
| | | VRTIRIALRRHGDAARIAYAFKPGAKRLKPGGGAQDHTPETH | | |
| | | ADAILEALLRWHELATGARWRDPWAETQWKDWVQPHISA | | |
| | | TLPELANDADRWERKRHRAALEQVLRPVAQMLIQRPTDAL | | |
| | | HQVWSKHWADEDLKWPSRLRWLRNWLLPRGPRARSGAA | | |
| | | RNVGGLSLLRIATLRELYQTQKAYAMRPEPDDPRKRIAGRN | | |
| | | DDRYDELGRSVLQVIERLREQRVKQLASRIVEAALGVGRAKP | | |
| | | TRGRQRPQSRVDVPCHAVIIESLRNYRPDELQTRRENRAIM | | |
| | | NWSAGKVRKYLEEACQLHGLHLREVMPNYTSREDSRTGLP | | |
| | | GVRCVDVPVDPKLGKPKAYWWNSVLSTARKKSIGDAASHD | | |
| | | KQGDATSRFIVELAGCLDRLKADGKPLPKTVRVPRIGGDLFV | | |
| | | AAPPTSCTAPAHQPHPACDGARALQADLNAAANIGLRALL | | |
| | | DPDFPAKWWYVPCIDDQRGLALPRADKVLGSACFPGDPAT | | |
| | | FGSLLKTRTAAGPAVDGQAAPDRKPRTGTHRPGSAKSRSLG | | |
| | | DGKATTNYWSDRSARDLRPADEGGHWQPTNVYWNWVR | | |
| | | KRALLGLYSFNGLSPPSDDRP | | |
| | | [SEQ ID NO: 19] | | |

### Example 3: Vector Cloning of MADZYME and PCR for In Vitro Test

The MADzyme coding sequences were cloned into a pUC57 vector with T7-promoter sequence attached to the 5′-end of the coding sequence and a T7-terminator sequence attached to the 3′-end of the coding sequence.

First, Q5 Hot Start 2× master mix reagent (NEB, Ipswich, MA) was used to amplify the MADzyme sequences cloned in the pUC57 vector. The forward primer 5′-TTGGGTAACGCCAGGGTTTT [SEQ ID NO: 55] and reverse primer 5′-TGTGTGGAATTGTGAGCGGA [SEQ ID NO: 56] amplified the sequences flanking the MADzyme in the pUC57 vector including the T7-promoter and T7-terminator components at the 5′- and 3′-end of the MADzymes, respectively. 1 μM primers were used in a 10 μL PCR reaction using 3.3 μL boiled cell samples as templates in 96 well PCR plates. The PCR conditions shown in Table 2 were used:

TABLE 2

| STEP | TEMPERATURE | TIME | |
|---|---|---|---|
| DENATURATION | 98° C. | 30 | SEC |
| 30 CYCLES | 98° C. | 10 | SEC |
| | 66° C. | 30 | SEC |
| | 72° C. | 3 | MIN |
| FINAL EXTENSION | 72° C. | 2 | MIN |
| HOLD | 12° C. | | |

### Example 4: gRNA Construction and PCR for In Vitro Test

Several functional gRNAs associated with each MADzyme was designed by truncating the 5′ region, the 3′ region and the repeat/anti-repeat duplex (see Table 3).

TABLE 3

| gRNA | | | | | |
|---|---|---|---|---|---|
| name | sgRNAv1 | sgRNAv2 | sgRNAv3 | sgRNAv4 | sgRNAv5 |
| sgM293 | GGGAAGGGCCTA | GGGAAGGGCCT | GGGAAGGGCCTA | GGAAGGGCCTAT | GGTCCAAGGGA |
| | TTTCCCAGCATGT | ATTTCCCAGCAT | TITCCCAGCATGT | TTCCCAGCATGT | AGGGCCTATTTC |
| | GTCTTCGCATTTA | GTGTCTTCGCATT | GTCTTCGCATTTA | GTCTTCGCATTTA | CCAGCATGTGTC |
| | ATTGCTTTAGCAC | TAATTGCTTTAGC | ATTGCTTTAGCAC | ATTGCTTTAGCA | TTCGCATTTAATT |
| | TGGGCATCGTTCT | ACTGGGCATCGT | TGGGCATCGTTCT | CTGGGCATCGTT | GCTTTAGCACTG |
| | TITTAGTAGTGTTT | TCTTTTTAGAAAA | AAAAAGACCGAT | CTTTTTAGAAAA | GGCATCGTTCTTT |
| | AAGTAGATACTAC | CTACTGAAAAGA | GGACACA | CTACTGAAAAGA | TTAGAAAACTAC |
| | TGAAAAGACCGAT | CCGATGGACACA | [SEQ ID NO: 24] | CCGATGGACACA | TGAAAAGACCGA |
| | GGACACA | [SEQ ID NO: 23] | | [SEQ ID NO: 25] | TGGACACA |
| | [SEQ ID NO: 22] | | | | [SEQ ID NO: 26] |
| sgM294 | GCCCCGATTTCCC | GCCCCGATTTCC | GATTTCCCTTTGA | GGATTTCCCTTTG | NONE |
| | TTTGAATGATCTC | CTTTGAATGATCT | ATGATCTCGGCCT | AATGATCTCGGC | |
| | GGCCTCGTTGCCA | CGGCCTCGTTGC | CGTTGCCACTGAC | CTCGTTGCCACT | |
| | CTGACCGAATTCT | CACTGACCGAAT | CGAATTCTTCCGC | GACCGAATTCTT | |
| | TCCGCCTTTGGAA | TCTTCCGCCTTTG | CTTTGGAATTCCA | CCGCCTTTGGAA | |
| | TTCCAAGCTCTTT | GAATTCCAAGCT | AGCTCTTTGACAT | TTCCAAGCTCTTT | |
| | GACATCGCGAGCC | CTTTGACATCGC | CGCGAGCCCGCG | GACATCGCGAGC | |
| | CGCGATGGGAAA | GAGCCCGCGATG | ATGGGAAAGAGA | CCGCGATGGGAA | |
| | GAGATTGTGGCG | GGAAAGAGATT | TTGTGGCGG | AGAGATTGTGGC | |
| | G | GTGGC | [SEQ ID NO: 29] | GG | |
| | [SEQ ID NO:27] | [SEQ ID NO: 28] | | [SEQ ID NO: 30] | |
| sgM295 | GGGAAGGGCCTA | GGGAAGGGCCT | GGGAAGGGCCTA | GGGCCAAGGGA | GGGCCAAGGGA |
| | TTTCCCAGCATGT | ATTTCCCAGCAT | TTTCCCAGCATGT | AGGGCCTATTTC | AGGGCCTATTTC |
| | GTCTTCGCATTTA | GTGTCTTCGCATT | GTCTTCGCATTTA | CCAGCATGTGTC | CCAGCATGTGTC |
| | ATTGCTTTAGCAC | TAATTGCTTTAGC | ATTGCTTTAGCAC | TTCGCATTTAATT | TTCGCATTTAATT |
| | TGGGCATCGTTCT | ACTGGGCATCGT | TGGGCATCGTTCT | GCTTTAGCACTG | GCTTTAGCACTG |
| | TITTAGTAGTGTTT | TCTTTTTAGTAAA | TAAAAAAGACCGA | GGCATCGTTCTTT | GGCATCGTTCTT |
| | AAGTAGATACTAC | AACTAAAAAGAC | TGGACAC | TTAGTAAAAACT | AAAAAAGACCGA |
| | TAAAAAGACCGAT | CGATGGACAC | [SEQ ID NO: 33] | AAAAAGACCGAT | TGGACAC |
| | GGACAC | [SEQ ID NO: 32] | | GGACAC | [SEQ ID NO: 35] |
| | [SEQ ID NO: 31] | | | [SEQ ID NO: 34] | |
| sgM296 | ATGTAACCCTATA | GGATGTAACCCT | GGTAACCCTATAG | GGGATGTAACCC | GGGTAACCCTAT |
| | GGGGTTGCGTGA | ATAGGGGTTGCG | GGGTTGCGTGAG | TATAGGGGTTGC | AGGGGTTGCGTG |
| | GTCGGCCATAGTG | TGAGTCGGCCAT | TCGGCCATAGTGC | GTGAGTCGGCCA | AGTCGGCCATAG |
| | CCTCGGCAACAGC | AGTGCCTCGGCA | CTCGGCAACAGCG | TAGTGCCTCGGC | TGCCTCGGCAAC |
| | GTAAAAAACTGCT | ACAGCGTAAAAA | TAAAAAACTGCTG | AACAGCGTAAAA | AGCGTAAAAAAC |
| | GCCAGTGGTCGAA | ACTGCTGCCAGT | CCAGTGGTCGAAG | AACTGCTGCCAG | TGCTGCCAGTGG |
| | GTAAGTCAACAAA | GGTCGAAGTAAG | TAAGTCAACAAAA | TGGTCGAAGTAA | TCGAAGTAAGTC |
| | ACGGAGGTGGTG | TCAACAAAAATG | ATGTAGGCCTACT | GTCAACAAAAAT | AACAAAAATGTA |
| | AAGTCACCCCCGT | TAGGCCTACTGG | GGCAC | GTAGGCCTACTG | GGCCTACTGGCA |
| | TTTGTAGGCCTAC | CAC | [SEQ ID NO: 38] | GCAC | C |
| | TGGCAC | [SEQ ID NO: 37] | | [SEQ ID NO: 39] | [SEQ ID NO: 40] |
| | [SEQ ID NO: 36] | | | | |
| sgM297 | CGGAGGGAACTC | GGAGGGAACTCC | GGAGGGAACTCC | GGCAGGCGGAG | GGCAGGCGGAG |
| | CGTGAACGTGTCT | GTGAACGTGTCT | GTGAACGTGTCTT | GGAACTCCGTGA | GGAACTCCGTGA |
| | TCCCCTTCGATGG | TCCCCTTCGATG | CCCCTTCGATGGG | ACGTGTCTTCCCC | ACGTGTCTTCCCC |
| | GCTTGGCACACGG | GGCTTGGCACAC | CTTGGCACACGGG | TTCGATGGGCTT | TTCGATGGGCTT |
| | GGTCAATCGAGTT | GGGGTCAATCGA | GTCAATCGAGTTA | GGCACACGGGG | GGCACACGGGG |
| | GCCGTTGAAGGC | GTTGCCAAAAGG | TCTCGATCGACCG | TCAATCGAGTTG | TCAATCGAGTTA |
| | GGCGAATTCGCCG | CATCTCGATCGA | ACAC | CCAAAAGGCATC | TCTCGATCGACC |
| | GCATCTCGATCGA | CCGACAC | [SEQ ID NO: 43] | TCGATCGACCGA | GACAC |
| | CCGACAC | [SEQ ID NO: 42] | | CAC | [SEQ ID NO: 45] |
| | [SEQ ID NO: 41] | | | [SEQ ID NO: 44] | |
| sgM298 | TTGCCTCTACAGG | GCCTCTACAGGA | GGGCCTCTACAGG | GGGACATTGGCC | NONE |
| | AGGCGAGAATGC | GGCGAGAATGCC | AGGCGAGAATGC | TCTACAGGAGGC | |
| | CACGGCACGTGTC | ACGGCACGTGTC | CACGGCACGTGTC | GAGAATGCCACG | |
| | TTCCCCTTCAATG | TTCCCCTTCAATG | TTCCCCTTCAATG | GCACGTGTCTTC | |
| | GGCTTGGCACCGT | GGCTTGGCACCG | GGCTTGGCACCGT | CCCTTCAATGGG | |
| | GGAGTCGATCAGT | TGGAGTCGATCA | GGAGTCGATCAG | CTTGGCACCGTG | |
| | TTTGTGCCGGCGA | GAAAACTGATCG | AAAACTGATCGAC | GAGTCGATCAGA | |
| | AGGGTTGCCGAC | ACGGACAC | GGACAC | AAACTGATCGAC | |
| | GTCAGCACCAACC | [SEQ ID NO: 47] | [SEQ ID NO: 48] | GGACAC | |
| | TGATCGACGGACA | | | [SEQ ID NO: 49] | |
| | C | | | | |
| | [SEQ ID NO: 46] | | | | |
| sgM299 | GTCGCCTATAGGG | GTCGCCTATAGG | GTCGCCTATAGGG | GGTCGCCTATAG | GGGTCGCCTATA |
| | CGATACAACTCCG | GCGATACAACTC | CGATACAACTCCG | GGCGATACAACT | GGGCGATACAAC |
| | AGCATGTGTCTTC | CGAGCATGTGTC | AGCATGTGTCTTC | CCGAGCATGTGT | TCCGAGCATGTG |
| | CCCTTCAATGGGC | TTCCCCTTCAATG | CCCTTCAATGGGC | CTTCCCCTTCAAT | TCTTCCCCTTCAA |
| | TTGGCACTCGGCA | GGCTTGGCACTC | TTGGCACTCGGCA | GGGCTTGGCACT | TGGGCTTGGCAC |
| | TCGATCAAGCTCG | GGCATCGATCAA | TCGATCAAGCTCG | CGGCATCGATCA | TCGGCATCGATC |
| | CGTCGGCTGTCGG | GCTCGCGTCGGC | CGTAAAAACGCG | AGCTCGCGTCGG | AAGCTCGCGTAA |
| | GCCAAAGCCGCGT | TGTCGTCGGCCG | GCCTTGATCGATG | CTGTCGTCGGCC | AAACGCGGCCTT |
| | CGGCCGACGCGG | ACGCGGCCTTGA | GACAC | GACGCGGCCTTG | GATCGATGGACA |
| | CCTTGATCGATGG | TCGATGGACAC | [SEQ ID NO: 52] | ATCGATGGACAC | C |
| | ACAC | [SEQ ID NO: 51] | | [SEQ ID NO: 53] | [SEQ ID NO: 54] |
| | [SEQ ID NO: 50] | | | | |

To find the optimal gRNA length, different lengths of spacer, repeat: anti-repeat duplex and 3′ end of the tracrRNA were included. These gRNAs were then synthesized as a single stranded DNA downstream of the T7 promoter with a proper guide sequence added at the 3′-end of each gRNA. These sgRNAs were amplified using two primers (5′-AAACCCCTCCGTTTAGAGAG [SEQ ID NO: 57] and 5′-AAGCTAATACGACTCACTATAGGCCAGTC [SEQ ID NO: 58]) and 1 μL of 10 μM diluted single stranded DNA as a template in 25 μL PCR reactions for each sgRNA according to the conditions of Table 4.

TABLE 4

| STEP | TEMPERATURE | TIME | |
|---|---|---|---|
| DENATURATION | 98° C. | 30 | SEC |
| 12 CYCLES | 98° C. | 10 | SEC |
| | 66° C. | 30 | SEC |
| | 72° C. | 2 | MIN |
| FINAL EXTENSION | 72° C. | 2 | MIN |
| HOLD | 12° C. | | |

The target library was designed based on an assumption that the eight randomized NNNNNNNN PAMs of these nucleases reside on the 5′ end of the target sequence (5′-CCAGTCAGTAATGTTACTGG [SEQ ID NO: 59]).

### Example 5: In Vitro Transcription and Translation for Production of MAD Nucleases and gRNAs

The MADZYMEs were tested for activity by in vitro transcription and translation (txtl). A PURExpress® In Vitro Protein Synthesis Kit (NEB, Ipswich, MA) was used to produce MADzymes from the PCR-amplified MADZYME (Example 3) and also to produce the gRNA (Example 4) in a single reaction. In each well in a 96-well plate, the reagents listed in Table 5 were mixed to start the production of MADzymes and gRNAs:

TABLE 5

| | REAGENTS | VOLUME (μl) |
|---|---|---|
| 1 | SolA (NEB kit) | 10 |
| 2 | SolB (NEB kit) | 7.5 |
| 3 | PCR amplified gRNA | 0.4 |
| 4 | Murine RNase inhibitor (NEB) | 0.5 |
| 5 | Water | 3.0 |
| 6 | PCR amplified T7 MADZYME | 3.6 |

A master mix with all reagents was mixed on ice with the exception of the PCR-amplified T7-MADZYMEs to cover enough 96-well plates for the assay. After 21 μL of the master mix was distributed in each well in 96 well plates, 4 μL of the mixture of PCR amplified MADZYMEs and gRNA under the control of T7 promoter was added. The 96-well plates were sealed and incubated for 4 hrs at 37° C. in a thermal cycler. The plates were kept at room temperature until the target pool was added to perform the target depletion reaction.

After 4 hours incubation to allow production of the MADzymes and gRNAs, 4 μL of the target library pool (10 ng/μL) was added to the 10 μL aliquots of in vitro transcription/translation reaction mixture and allowed to deplete for 30 min, 3 hrs or overnight at 37° C. and 48° C. The target depletion reaction mixtures were diluted into PCR-grade water that contains RNAse A incubated for 5 min at room temperature. Proteinase K was then added and the mixtures were incubated for 5 min at 55° C. RNAseA/Proteinase K treated samples were purified with DNA purification kits and the purified DNA samples were then amplified and sequenced. The PCR conditions are shown in Table 6:

TABLE 6

| STEP | TEMPERATURE | TIME | |
|---|---|---|---|
| DENATURATION | 98° C. | 30 | SEC |
| 4 CYCLES | 98° C. | 10 | SEC |
| | 66° C. | 30 | SEC |
| | 72° C. | 20 | SEC |
| 12 CYCLES | 98° C. | 10 | SEC |
| | 72° C. | 20 | SEC |
| FINAL EXTENSION | 72° C. | 2 | MINUTES |
| HOLD | 12° C. | | |

### Example 6: PAM Identification

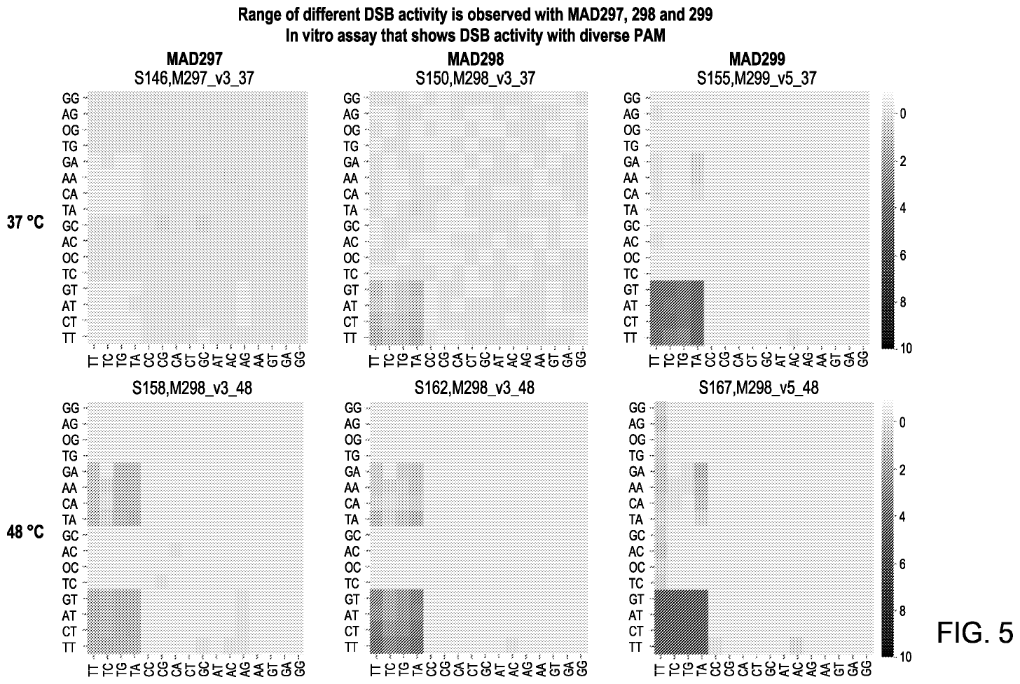

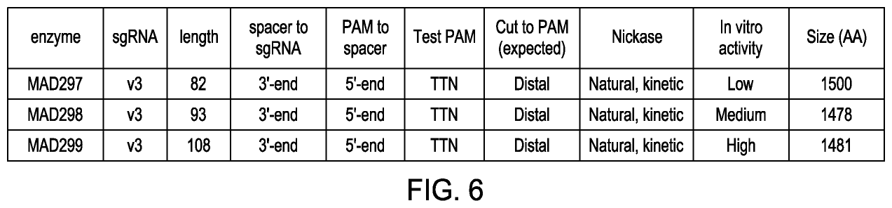

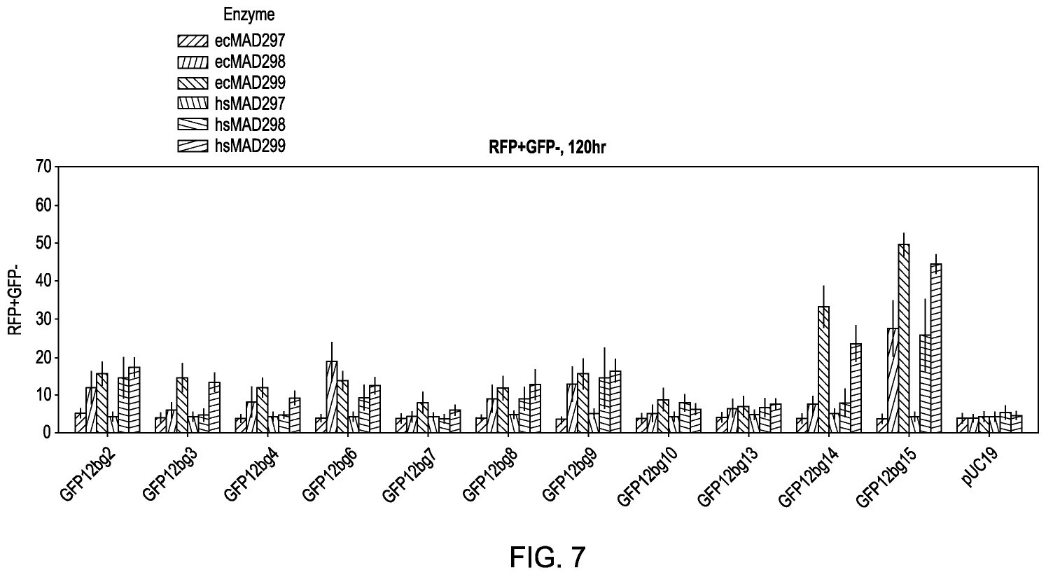

The MAD297, MAD298 and MAD299 were screened for double-stranded break activity. FIG. 5 shows that the identified active MADzymes, namely MAD297, MAD298 and MAD299, had a wide PAM preference and high activity. FIG. 6 shows various parameters for the MADzymes with activity.

### Example 7: In Vitro Plasmid Cut and Nick Assay

In vitro plasmid cut and nick assay with MAD297, MAD298, and MAD299 were performed by producing enzymes and sgRNA (sgM297v3, sgM298v3, and sgM299v5) with a guide sequence [SEQ ID NO: 59] attached to the 3′-end of each sgRNA under the T7 promoter in vitro. After the formation of RNP complex with sgRNA and MADzymes produced, supercoiled target DNA with an embedded target sequence [SEQ ID NO: 59] with the 5′-end addition of acgaTTTG replacing the randomized PAM sequence was added to the RNP complexes, with the knowledge that “TTTG” serves as an active PAM sequence for all three MADzymes. After 30 min incubation at 37° C., reaction mixtures were treated with RNase and Proteinase K then the target plasmid was purified using a PCR cleanup kit. The resulting plasmid targets were run on a TAE-agarose gel where different forms of plasmid architecture were visualized and distinguished between supercoiled, nicked, and linear DNA, where all three MADzymes (MAD297, MAD298, and MAD299) showed the nicked plasmid target product as the major product in vitro (data not shown).

### Example 8: In Vivo Activity Assay in Mammalian Cells

Two different codon optimized versions of MADzymes were cloned under the CMV promoter and expressed in mammalian cells. The testing host cell line was the HEK293T cell line with a single copy of synthetic target GFP locus integrated randomly in the genome. Corresponding guides targeting the GFP locus were all cloned under the hU6 promoter with one of the sgRNA scaffolds chosen from the Table 3 for each MADzyme (sgM297v3, sgM298v3, and sgM299v5) with a guide sequence (Table 7) connected to the 3′-end of each sgRNA and an additional hepta-T sequence as a translational stop for guide expression. Table 7 shows the guide sequences with the PAM and the direction related to the direction of target GFP expression.

TABLE 7

| guide | PAM | guide_seq | SEQ ID NO: | direction |
|---|---|---|---|---|
| GFP12bg2 | TTG | CCGGTGGTGCAGATGAACTTCAG | [SEQ ID NO: 60] | Rev |
| GFP12bg3 | TTG | AAGAAGTCGTGCTGCTTCATGTG | [SEQ ID NO: 61] | Rev |
| GFP12bg4 | TTG | CCGTAGGTGGCATCGCCCTCGCC | [SEQ ID NO: 62] | Rev |
| GFP12bg6 | TTC | GGGCATGGCGGACTTGAAGAAGT | [SEQ ID NO: 63] | Rev |
| GFP12bg7 | TTG | TGGCCGTTTACGTCGCCGTCCAG | [SEQ ID NO: 64] | Rev |
| GFP12bg8 | TTG | AAGAAGATGGTGCGCTCCTGGAC | [SEQ ID NO: 65] | Rev |
| GFP12bg9 | TTT | GCCGGTGGTGCAGATGAACTTCA | [SEQ ID NO: 66] | Rev |
| GFP12bg10 | TTC | ATCTGCACCACCGGCAAACTGCC | [SEQ ID NO: 67] | For |
| GFP12bg11 | TTC | AGCGTGTCCGGCGAGGGCGAGGG | [SEQ ID NO: 68] | For |
| GFP12bg12 | TTG | GTGACCACCCTGACCTACGGCGT | [SEQ ID NO: 69] | For |
| GFP12bg13 | TTC | AGCCGCTACCCCGACCACATGAA | [SEQ ID NO: 70] | For |
| GFP12bg14 | TTC | TTCAAGTCCGCCATGCCCGAAGG | [SEQ ID NO: 71] | For |
| GFP12bg15 | TTC | TTCAAGGACGACGGCAACTACAA | [SEQ ID NO: 72] | For |
| GFP12bg16 | TTC | AAGGACGACGGCAACTACAAGAC | [SEQ ID NO: 73] | For |

A guide expressing plasmid (50 ng, or pUC19 as a negative control) and a MADzyme expressing plasmid (50 ng) was mixed with 1 μL Polyfect and diluted in 35 μL OptiMem, and added to 25K TrypLE singulated HEK293T host cells as described above in 100 μL DMEM for reverse transfection. Five days after transfection, the cells were collected and analyzed using a flow cytometry to detect depletion of a GFP signal as evidence of double-strand breaks that interrupt the expression of the target gene. The results are shown in FIG. 7. As shown in FIG. 7, MAD298 and MAD299 showed evidence of double-strand breaks in vivo, where MAD297 did not.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112. ¶6.

We claim:

1. A nuclease system configured to perform nucleic acid-guided nuclease editing, wherein the nuclease system is selected from a MAD293 system comprising SEQ ID NO: 1 (MAD293 nuclease), SEQ ID NO: 2 (CRISPR RNA) and SEQ ID NO: 3 (trans-activating crispr RNA); a MAD294 system comprising SEQ ID NO: 4 (MAD294 nuclease), SEQ ID NO: 5 (CRISPR RNA) and SEQ ID NO: 6 (trans-activating crispr RNA); a MAD295 system comprising SEQ ID NO: 7 (MAD295 nuclease), SEQ ID NO: 8 (CRISPR RNA) and SEQ ID NO: 9 (trans-activating crispr RNA); a MAD296 system comprising SEQ ID NO: 10 (MAD296 nuclease), SEQ ID NO: 11 (CRISPR RNA) and SEQ ID NO: 12 (trans-activating crispr RNA); a MAD297 system comprising SEQ ID NO: 13 (MAD297 nuclease), SEQ ID NO: 14 (CRISPR RNA) and SEQ ID NO: 15 (trans-activating crispr RNA); a MAD298 system comprising SEQ ID NO: 16 (MAD298 nuclease), SEQ ID NO: 17 (CRISPR RNA) and SEQ ID NO: 18 (trans-activating crispr RNA); and a MAD299 system comprising SEQ ID NO: 19 (MAD299 nuclease), SEQ ID NO: 20 (CRISPR RNA) and SEQ ID NO: 21 (trans-activating crispr RNA).

2. The nuclease system of claim 1, wherein the nuclease system is a MAD293 system comprising SEQ ID NO: 1 (MAD293 nuclease), SEQ ID NO: 2 (CRISPR RNA) and SEQ ID NO: 3 (trans-activating crispr RNA).

3. The nuclease system of claim 1, wherein the nuclease system is a MAD294 system comprising SEQ ID NO: 4 (MAD294 nuclease), SEQ ID NO: 5 (CRISPR RNA) and SEQ ID NO: 6 (trans-activating crispr RNA)

4. The nuclease system of claim 1, wherein the nuclease system is a MAD295 system comprising SEQ ID NO: 7 (MAD295 nuclease), SEQ ID NO: 8 (CRISPR RNA) and SEQ ID NO: 9 (trans-activating crispr RNA).

5. The nuclease system of claim 1, wherein the nuclease system is a MAD296 system comprising SEQ ID NO: 10 (MAD296 nuclease), SEQ ID NO: 11 (CRISPR RNA) and SEQ ID NO: 12 (trans-activating crispr RNA).

6. The nuclease system of claim 1, wherein the nuclease system is a MAD297 system comprising SEQ ID NO: 13 (MAD297 nuclease), SEQ ID NO: 14 (CRISPR RNA) and SEQ ID NO: 15 (trans-activating crispr RNA).

7. The nuclease system of claim 1, wherein the nuclease system is a MAD298 system comprising SEQ ID NO: 16 (MAD298 nuclease), SEQ ID NO: 17 (CRISPR RNA) and SEQ ID NO: 18 (trans-activating crispr RNA).

8. The nuclease system of claim 1, wherein the nuclease system is a MAD299 system comprising SEQ ID NO: 19 (MAD299 nuclease), SEQ ID NO: 20 (CRISPR RNA) and SEQ ID NO: 21 (trans-activating crispr RNA).


SEQUENCE LISTING (73 sequences)

SEQ ID NO: 1 - protein, 1400 aa, Artificial Sequence
  ENGINEERED NUCLEASE
MAAFQRSYTMNLKPATSEQDKFILWNRLFLTHWSVNEGAKIFGELFLNLRGGLSPELDIFDLDKVKDDKKKKAFVMGRRRLLALGWLSVEDNLSAGEHPFRIREIPVGRNMGKSQASTLLTEILKNKGIKDEAVIKEWIDDCTPSLIANIREDAVWINRASSFNSITPCPTKDEVWIVLSGLLGLRFLDLSLEEVKGKETEYLYDLGEKETQSKSDPSKKARELFGNLFTQNPVLMKNSRDKKDTFAKEFYLAFKEFKDYEKLKEKIESWRKEKEFPLIENPVAEKYPPEVTFTGSPCTVSKRYRKLLVSLELWPSSQDENGNIPKTEKTEDKTHNQVLLDYLLKACNEGNKGTQKIITPVWANNLKAELELKMNEIIRIGESSSTELQRLMIKMAARRISQTLSWIKINEQTKHDAYQKKNKAFKLLSEIDKNGEACKWLENYELFRTDDSGGEEYHISLRAISCWKQILEEWQKNDSPKALREKVKEVQAEEEKFGDARLFEDLADDNARSVWLLPDGNKTPDILNWWCEYRTAEIDESRFKIPCYCHPHPFKHPVYVEYGKSNPKVIFAMKNNKVKKGHIEHGWNPQNPRSIALSLFNNGNRESSLVPFIWESKRLWKDLGGEATQIGDIPRSDRMGLSGKRESVKPKAPFQKEVWNARLQSDRRTLEKLEKYWNPESMKWIDDGKFLIQSKWFITFGPDMETAEGPWKLYLKEKYVDNKILGNRSKENQKRGYRAKKLLSGYPAGMRILSVDLGHRYAASCAVWETITKKQITEELAYQPDNNSLFEHSCKTIDKKIKNTVYRRIGEDSIDAPWAKLEKQFTIKLQGEDKSCYLLRSDEKELFRSILSKLSCLNNDTGHNILEMIENLLRIVKAKIYRQGILARISYSMTAQYKPGKGGQKSPLSDEDKIHYLSENLAAWSAIMGNQEWNEDVISDWYKTYISHLVSGPKPKEGNRKSDRDKIIEYFLPAARKLYDDNETRIKIHDLFKELWDENNKQLSAVLKEIKKIILPKGIRYFDKNTDNPSKWKNNQSKLKQITHRGGLSMQRIVAIEEYYKLAKAYKNHPEPDDLTKNIPLPGDNSSAGFNQRIRDTLERMKEQRVKQIASRIVESALGLGIEGYKKRPLTPENKPCQAIVIEDLSHYRPDELQTRRENRRLMQWSSSKVKKYLKEACEMHDVRLVEISPEYTSRQDSRTGAAGLRCIDINIREFLKDSSRWQNKINTIQKKPANKKSNLDQYLIELNESLGNKYKDKVIPSDNFVRIPRKGGDVFVSSSKESPVSKGIQADLNAAANIGLKALLDPDWAGAWWYILIEVKSNHVIPYGEKYKGSECLRAWKFSGLENQVMKNNMNLWRDLQSQFSGEDKWMSYKEYNELTEKRVINILRERAGLELIKE

SEQ ID NO: 2 - DNA, 37 bp, Artificial Sequence
  CR RNA FOR ENGINEERED NUCLEASE
gtttaagtagatactactgaaaagaccgatggacaca

SEQ ID NO: 3 - DNA, 74 bp, Artificial Sequence
  TRACRRNA FOR ENGINEERED NUCLEASE
gggaagggcctatttcccagcatgtgtcttcgcatttaattgctttagcactgggcatcgttctttttagtagt

SEQ ID NO: 4 - protein, 1375 aa, Artificial Sequence
  ENGINEERED NUCLEASE
MNRIYQGRVSKIEIKDSEGNFRNVPVGSPDTCPLWRHHRIFQDAVNYYLVALGALAGTGSENAFVGLGSKDRVIHDLYSRLFDSWERFPRDMHGASSLRDSLRRTLPGLSERASLQDAFDAILSGNEANARERVLSLLSLIQDLGGDIQKGSKRYFPFFCEPATKATFPRARVGLLKVEGKDFVPRLLWSSDLEIAPDQVVEQLKFEYFANPNESVQPIEGNEARVRLIEALDNPQLGIELPIEILSDLRKRVHLIETDIRIPRYFFGGAGAELRKFRLDLFLIAAYVTPDPSILRALRNSFKEPSASKSSKKKDETEEVENLLRSLGDDPLILARGERGFVFPSFTSLPTWVGANAQKPIWRDFDIAAFAEALKSLNQFTAKTEEREEKLKKAEETLHYMLGISDAIPRSSDSETEEQAPSRPGKDPRWPLVAQLEKELGENLSEGTWQLSRSAMRGLRDIIGLWRKHPGASVVTLQKDVKTYQADEKHKREIGSVQLFLLLCEERYHALWQTETDDERGDESEENDDPARILSDAIEVHQIRREVERFREPIRLTPAEPVFSRRLFMFSDLTDKLAKVKFGETTEENSEVKSQFVEAAIALKEGENLKEARVRITFSAPRLHRDELLGGAESRWLQPITAALGFSNPAPSVKFDSAVALMPDHMDDGRIRHLLNFPVNFDSAWLHQSIGKADLWKSQFNGTKDKNLHLHWAGTARDTTRKNTWWENRTIIENGFTVLSNDLGQRSAGAWALLKVTCSRPDTKHPVRSIGHDGTREWFATVLATGIHRLPGEDQRILKNGKWATEQSGKKGRNATFSEYEAACVLAKNLGCESVENWLGMSGEKSYPALNDQLVKIANRRITRLGTYHRWSCFSPEKFEDPARRANVIGGQLAELSAYQDENVTVSADILKSGDFEGFRHRAGAAFEALRTELEVHLVNLANLTAPLRQKVWSWQKRPDSSGYGDLLMVDLDDCHPKIRGQRGLSMARLEQLEGLRRLFLRYNRSLDRSPGIPAKFGREDVGRTSGEPCQALLVKIDRMKEQRVNQTAHLILAQALGVRLCPHRIEENERKSRDLHGEYEKIPGREPVDFIVIEDLSRYLSSQGRAPSENSRLMKWAHRAVRDKLKMLAEEPFGIPVVETVPAYSSRFHALNGQAGSRLHELHELEAYQQQSLINLAAKTDFQNRDRSKAAGELFEQFQALAKLNERRRAEGKKVPRTLYYPKSGGPLFLASRDGDTIHADVNAAINLGLRAIAAPACIDIHRRLRATKEKEVYRPRVGNAREKSAFSKDDIIQPSGAPSKKFASSSSPNFFYEPEDLKQANGEPLFDRAMFGEYSLVSGVSLWSMVNNAIYIRCVELNRTRLHGKDPDDQIPM

SEQ ID NO: 5 - DNA, 36 bp, Artificial Sequence
  CRRNA FOR ENGINEERED NUCLEASE
gccgcagcccccgcgatgggaaagagattgtggcgg

SEQ ID NO: 6 - DNA, 97 bp, Artificial Sequence
  TRACR RNA FOR ENGINEERED NUCLEASE
gccccgatttccctttgaatgatctcggcctcgttgccactgaccgaattcttccgcctttggaattccaagctctttgacatcgcgagcgctgagg

SEQ ID NO: 7 - protein, 1397 aa, Artificial Sequence
  ENGINEERED NUCLEASE
MAAFQRSYTMNLKPATSEQDKFILWNRLFLTHWSVNEGAKIFGELFLNLRGGLSPELGIFDLNKDKDDRKKKALVMGRRRLLALGWLSVEDNLSAGDHPFRIREIPVGRNMEIKQATTLLTEILKNKGVKDEAVIKEWIDDCTPSLIANIREDAVWINRAKSFYSMNPCPTKDEVWKILSYVLNTSFLDLSLNDSSERDNTKNKKGTKENEKDVSNKSKELYGWLFTKNPNKMREAGENKDKFINNFRENFNTFTDYANLKVEIELWRKNNISNTLLITQKAKYPPEVKEANHPSKFSVGYRKLLVHLELWPSSKDENGDIPKGIEGKDKSHNQILLDYLLEVCNEGNKTTKKVIVPAWADGIKTELESKASIKVGDSTSSVLQRLMIKMAARRISQTLSWIKINEQVRHDAYQKKNKAFKLLCEIDKNGEACKWLENYELFRRDDSGGEEYHISARAISCWKQILEEWQKNDSSKALREKVKVVQAAEDKFGDARLFEDLADDNARSVWLLPDGNKTPDILNWWCEYRTADIDESRFKIPCYCHPHPFKHPVYVEYGKSNPQVIFSLKHDKARKNRIDNGWNPKNPRILALLLLDIVRQKSTLAPFVWESKRLWKDLGGDATVTYKIPRSDRMGLSSIGNIDYARPEVPFLKEKWNARLQSDRRTLEKLEKYWNPESMKWIDDGKFLIQSKWFITFGPDMETAEGPWKLYLKENINDNNYLGNRSKENQKRGYRAKKLLSGYPAGMRILSVDLGHRYAASCAIWETITKKQITEELAYQPDKNSVFEHSCKTIDKKIKNTVYRRIGDDSIDAPWAKLEKQFTIKLQGEDKSCYLLRSDEKELFRSILSKLSCLNNDTGHNILEMIENLLRIVKAKIYRQGILARISYSMTAQYKPGKGGQKSPLSDEDKIHYLSENLAAWSALMGNQEWNEDGISDWYKKYISHLVSGPKPKEGNRKSDRDKIIEYFLPAARKLYDDNETRINIHDLFKELWDENNKQLSAVLKEIKKIILPKGIRYFDKNNDSSSRWKNNQSKLKQITHRGGLSLRRIVAIEGYYKLAKAYKNHPEPDNLTKNIPLPGDNSSAGFNQRIRDTLERMKEQRVKQIASRIVESALGLGIEGYKKRPLTPESKPCQAIVIEDLSHYRPDELQTRRENRRLMQWSSSKVKKYLSEACEMHDVLLVEISPEYTSRQDSRTGVAGLRCIDINIREFLKDSSSWQNKIKTIQMKPTNKKSNLDQYLIELNESLGERYKDKVIPSDKFVRIPRKGGDIFVSSSKESPVSKGIQADLNAAANIGLKALLDPDWAGAWWYILIEAKSNHVIPYGKKYKGAECLRDFKFSGLENQVMKNNMNLWRDLQSQFSSEDKWMSYKEYNELTEKRVINILRERAGLELIEE

SEQ ID NO: 8 - DNA, 36 bp, Artificial Sequence
  CR RNA FOR ENGINEERED NUCLEASE
gtttaagtagatactactaaaaagaccgatggacac

SEQ ID NO: 9 - DNA, 74 bp, Artificial Sequence; identical to SEQ ID NO: 3
  TRACR RNA FOR ENGINEERED NUCLEASE

SEQ ID NO: 10 - protein, 1097 aa, Artificial Sequence
  ENGINEERED NUCLEASE
MVTRALNLKLVVPRRPGELTKAEALWSTHDIVNRATSYYESQLLLCRQQDYQTRELTVSAGDQAPDLDALIANARDRNRYRGLEKPQVVREKLRNLYEAIVPPAIGKTGTAQAVGAFVSPLLDADSRGFTEIFDKIEALPNWVDGVRAEEPDALEAAADWLKSPQGKERLRPTGAPPTWIKLAKKKDAGWAAAFVADIDKKLKEVEGTPTLMQELRALGVMPLFPSFFASRIAGHKGAVSTWDRLALRLAVAHLLSWESWVELAAKEHAARVAKLEKFRDDNILGEIADAVEALRLYEKERTEELQQKAQLDAEEVRTTSRTIRGWVDLREKWLKTDASPDALISLVAAEQKRKSGKFGDPQLFRWLAKPENHFVWNKPDFDPPSLFASLRMIEGLVERSKETAWMTLPDARLHPRSSQWEPHGGGNLKTFRLEQGEGGSLSVTLPLLRKSGDDSYVEEEHAFSLAGSKQIPNASLDVRRNKYCLSYRTPTGEEAEAVVGSADLLLDWYFLQQRSEHRPEEGDIGPAFLKLALDITPIDPVWGEREKTPAIHHFKTASGKNTRHADGVAPGFRMLAVDLGIRTLATCSVFELKATAPAGRLSFPIAHLDLHAVHERSFTLTLDGEDPDRDAERWRENKSAELRRLRMGLTRYRNIRNMREDAPDEREVLLEDLQEKVQEHGWAFEEPLLRELAKHKDTPEPIWEAELTKALAQFRSDFGVIVGEWRRSNRARSTDSHAGKSMWAIDHLTNSRRFLMSWSLLSKPGQIRRLDRDKQGVFAKHLLDHLEGLKADRLKTGSDLIVQAARGFRRDKRGNWHKAYKPCHGILFEDLSRYRMRTDRPRRENSQLMKWAHRAVPKEVGMQAEVYGIRVEDTGAAFSSRFHAASHTPGIRMHPICQKDLENEWLLDEIEKQNSGVKRRELKLGQLVQLNGGELFACVTASGVKTLHADINAAQNLQRRFFTRHGDAFRIVARKVLVDEEEVWVPRSLGKRLLGALGSHGKLVPTGHESGSCRFEEITTRAWSKLSGEKLSDDRVGNEEDQIIASIEEEALERTGEVVVFFRDPSGQVLPRDLWYPSKTFWSIVKSTTLSKLKAAP

SEQ ID NO: 11 - DNA, 36 bp, Artificial Sequence
  CR RNA FOR ENGINEERED NUCLEASE
ggtgaagtcacccccgttttgtaggcctactggcac

SEQ ID NO: 12 - DNA, 98 bp, Artificial Sequence
  TRACR RNA FOR ENGINEERED NUCLEASE
atgtaaccctataggggttgcgtgagtcggccatagtgcctcggcaacagcgtaaaaaactgctgccagtggtcgaagtaagtcaacaaaacggaggt

SEQ ID NO: 13 - protein, 1500 aa, Artificial Sequence
  ENGINEERED NUCLEASE
MATAINYPTTQRAYTLRLRGIDPQDQSWRDALWATHEAVNRGAKVFGEWLLTLRGGLDHQLADAPVKVRGGTTRLPSDEERRDRRVLLALSWLSVEDAHGAPPDASLIVAKGTDSADCRARKLADALIAILQARSVAASEIGDPSKPPEDQPGTWLGDCMGSLSAAIRDDAVWVNRSKAFDAATQSCPSLTRDEIWDFLEPFFASPDAYLKPERAESDEGDSTSAATEDKAKDLVQKAGGWLSKRMGAGGGANFQDLARAYQAIAQWASSAQPGQSAQQAVGSLAGYLSQHGFSPTANDATGVLAVISGPGYKSATRNHITAIATSPEITPQDLSKLQELATKDKAGCSSKIGGKGPRPYATMILQQVEAACGFTYLQSDGPARHREFSVMLDHAARRVNVAHSWIKNAEAERRQFESDARRIKKVPQDALNWLRGYCEERGGASGSLEGYRIRRRAIDGWDQVVIRWSRSDCQSADDRIAAARQLQDDPEIDKFGDIQLFEALAAEEALCVWKPDGNPTAQPLKDFVAATEADAKKKRFKVPAYRHPDPLRHPVFTDFGNSRWGIEYSAHRAPAKCDELGQQVDRLTQAVAEAQRNLDGATAAQRASRESKLAEAQSKLVAAQTEFAAINDPHRVELKLWNGQAVAAIPMRWSSKRLIADLSLRRATQPSSDQRIGVTRADRLGRAAGNADDGRPVTITGLFQQDHWNGRLQAPRAQLDAIAKHVDKHGWDAKARRQIARIRWVVSFSAELSQQGPWFEFCHRFGEDAPARPFVSRHGEYAVKHRDNDQRKGHAKLILSRLPGLRVLAVDLGHRYAAACAVWEAISSDQMRQACAAANAPAPHPLAMYIHLKSTTAKGKPTTTIYRRIGPDKLPDGTPHPAPWARLDRQFLIKLPGEDRPARAASPDEIKAVEDFEDSVGRVRTAVDPPRKRGVDLLMHDAVRTARLALARHGRRARIAFQLISQVRILPGGRPQTLDDAGRRDLLNDTLADWYALATDSRWTDAAARQLWNERLAALNGGFTIDPPADASQPEAERTRAQRRQAEQELRHRLASLVEALFCNPTLCQQLHQAWTDRWNADDQQWRSRLKWLSRWLLPRGGSRRDGSRRHVGGLSLTRISTLIDFRRKVQVGYFTRLRPDGSRAEIGPQFGQSTLDAIQRLKDQRIKQLTSRIVEAALGIGVEQDRIWDAAKRKWRTVKRPREPRYHVDDQGVQQRDPRFQACHAVVIEDLSHYRPEETRTRRENRATMDWKSAETRKRLADHCQLYGLHLRDVNPQYTSRQDSRTGAPGCRCVDVSVADFLTKPAWRKQVAQARGKVASNRGDARDRLLVELDHQLTTANSLRGEMDSLRIPVNGGEVFVSADPRSPLAAGIQADLNAAANIGLRALMDPDFLGTWWYVPCDPSTKKPHIEKVKGSILATVGALQATSEEAAAPPRRGRGGTRSAAPREVINLWRDPSAVRIQDATAGEVWDVTPVYWSIVKDRVVDVLRQRNTKSGD

SEQ ID NO: 14 - DNA, 36 bp, Artificial Sequence
  CR RNA FOR ENGINEERED NUCLEASE
ggcggcgaattcgccggcatctcgatcgaccgacac

SEQ ID NO: 15 - DNA, 73 bp, Artificial Sequence
  TRACR RNA FOR ENGINEERED NUCLEASE
cggagggaactccgtgaacgtgtcttccccttcgatgggcttggcacacggggtcaatcgagttgccgttgaa

SEQ ID NO: 16 - protein, 1479 aa, Artificial Sequence
  ENGINEERED NUCLEASE
MSQQVKPPVTQRAYTLRLRGIDPSDTSWRKALWQTHEGVNKGAKAFGDWLLTLRGGLDHTLADTKVKGGKGKPDRDPTDEERKARRILLALSWLSVESKLGAPVGFIIASGTEAAEDRNRKVVAALEEILKSRNVATNEIDQWKNDCSASLSAAIRDDAVWVNRSKAFDEAVKSIGSSLTREEAWDMLERFFGSRDAYLAPAKGSEDESSETEQEDKAKDLVQKAGQWLSSRFGTGKGADFCRMANVYGKIAAWADNAQADTTGNDAINNLAAALNEYSPEPNDLKGVLGLISGPGYKSATRNLLNQLAAKATVTQQDFVSLKDKASNDAQKCKQNTGSKGPRPYSDAILKNVESVCGFTYLQDGGPARHSEFAVILDHAARRVSLAHTWIKRAEAERRKFEEDAKKIDQVPKAAKDWLDSFCLERSGASGALEPYRIRRRAVDGWKEVVAAWSKADCKTAEDRIAAARALQDDPEIDKFGDIQLFEALAEDDAVCVWHKDGDAAKPPDPQPLIDYALAAEAEFKKRHFKVPAYRHPDALLHPVFCDFGNSRWDICFDVHKNVQTPFPRALSLTLWTGSGMVSVPLCWQSKRLARDLALGQNAQNDGSSEVTRADRLGRAASNVTKSDEVKISGLFEQEDWNGRLQAPRQQLEAIAAVRDNLSLSDQERERRMSGMIDRIRWLVTFSAKLQPQGPWCEFAEKIQIGINPQYWPHADTNKDRKGHARLILSRLPGLRVLSVDLGHRYAAACAVWEAVNAEQINVACRAAGHREPKASDLYLHLKRKTTKQKKGDQVEFKETTIYRRIGADTLPDGTPHPAPWARLDRQFLIKLQGEEEGVRAASNEEIWAVHRLEVELGRTAPLIDRLVKAGWGQSGKQKTRLDALRNLGWAPANEVQGSDEMDEGEVHKPSLSVDELMSSAVRTMRLALKRHGDRARIAFAMTAAYKPMPGDRKYYFTEAKDASANEDATARNGKHIEFIQDALLLWYGLTSSRGWRDDAARQLWDDHIAKLSVYKAPEEIGEDASGVERKTKQKENREKLHDVAKALAQDVNLRKVLHNAWKKRWEKDDERWKKQLRWFKDWVFPRGKHASDPAIRKVGGLSLPRLATLIEFRRKVQVGFFTRLQPDGTRAETKEQFGQSALDTLEHLREQRVKQLASRIVEAALGIGRVRRPLGGKDPKRPDVRVDEPCHAIVIEDLTHYRPEETRTRRENRQLMTWSSSKVKDYLSEACQLHGLHLREVSASYTSRQDSRTGSPGIRCQDVPVKEFMRSPFWRKQVAQAEKKGDAHERFLCELNAMWKDKTVADWEKAGAVRVPLKGGEVFVSADRISPSAKGLQADLNAAANIGLRALTDPDWPGKWWYVPCEPASFRPAKDKVDGSAVVNPGQPLRQSAQAQSGDAAKDKKKRGNKGAGQSKEVVNLWRDISSSPLECIECGEWKEYAAYQNEVQYRVIRILEEQIKGRDRQPHEGSREDDIPF

SEQ ID NO: 17 - DNA, 36 bp, Artificial Sequence
  CR RNA
gttgccgacgtcagcaccaacctgatcgacggacac

SEQ ID NO: 18 - DNA, 93 bp, Artificial Sequence
  TRACR RNA FOR ENGINEERED NUCLEASE
ttgcctctacaggaggcgagaatgccacggcacgtgtcttccccttcaatgggcttggcaccgtggagtcgatcagttttgtgccggcgaagg

SEQ ID NO: 19 - protein, 1481 aa, Artificial Sequence
  ENGINEERED NUCLEASE
MARRSASTSKPPGPVAPTTQRAYTLRLRRAPGKCPHCEQDACDCWREALWATHAAFNRGAKAFGDWLLTLRGGLSHELAEQPSPPKNKEPTCEESDAIRKNRRILLALSWLSVEDDCSAPTGTFRVASGKDSEAERKNKVLTAFRSILTARRMRSQDVESWIADCAASLSAKIREDAVWINRSACFDQRALDLKVLSREYAKAAVMSFFGPLDEYFKLPDEADDTKPAVGGDGPDFRTLARQWVSTNFGTGKKSDSEAIAQNLRKLADANLAPFSGKPKAALIAHLSVELDGSTADIDGLCRAIGWNTGRPSKGRVAIERLPDPPTETSIQTMQQKFREEAEAKASSKGLRQVPEWMPAFQKSIERDCGMPFKLGEGRDHIGEFSVMLDHAARRVSIGHSWIKRAEAERRRFEADAQRLNHIPAAAKDWLDQFVQFRSGSSGAAAAGGEYRIRRRAIEGWDEIIKRWKRAACKSPEDRVAAAREVQADPEIEKFGDIQLFEALAADDAECVWRGDGNGTPDPLKDYVAATDALDKMRRFKVPAYRHPDPLAHPVFGDFGNSRGDIRFAVHEAAKATRGTKRIAKDQKEWIRERHGLRMGLWDGQSVRTADLRWSSKRLVDDLALRNHVTTRRTGPVSRADRLGRAAAGLGADEAACVAGLFELPDWNGRLQAPRAQLDAIAACVAANGGKWDDKARKLRDRIEWLVSFSAKLECCGPFMEYASQNGIQPNGKGEYYPHAERNKGRTGHAKLILSRLPGLRVLAVDLGHRFAAACAVWEALSKIAFDAETKGREVVSGGRAADDLYCHTRHLDCAGKARTTIYRRIGPDKLPDGSDHPAPWARLDRQFLIKLQGEERPARAAGPAETAAVQQIETDLGRARGQEDLPPRPVDSLMREAVRTIRIALRRHGDAARIAYAFKPGAKRLKPGGGAQDHTPETHADAILEALLRWHELATGARWRDPWAETQWKDWVQPHISATLPELANDADRWERKRHRAALEQVLRPVAQMLIQRPTDALHQVWSKHWADEDLKWPSRLRWLRNWLLPRGPRARSGAARNVGGLSLLRIATLRELYQTQKAYAMRPEPDDPRKRIAGRNDDRYDELGRSVLQVIERLREQRVKQLASRIVEAALGVGRAKPTRGRQRPQSRVDVPCHAVIIESLRNYRPDELQTRRENRAIMNWSAGKVRKYLEEACQLHGLHLREVMPNYTSREDSRTGLPGVRCVDVPVDPKLGKPKAYWWNSVLSTARKKSIGDAASHDKQGDATSRFIVELAGCLDRLKADGKPLPKTVRVPRIGGDLFVAAPPTSCTAPAHQPHPACDGARALQADLNAAANIGLRALLDPDFPAKWWYVPCIDDQRGLALPRADKVLGSACFPGDPATFGSLLKTRTAAGPAVDGQAAPDRKPRTGTHRPGSAKSRSLGDGKATTNYWSDRSARDLRPADEGGHWQPTNVYWNWVRKRALLGLYSFNGLSPPSDDRP

SEQ ID NO: 20 - DNA, 36 bp, Artificial Sequence
  CR RNA FOR ENGINEERED NUCLEASE
gccgcgtcggccgacgcggccttgatcgatggacac

SEQ ID NO: 21 - DNA, 94 bp, Artificial Sequence
  ENGINEERED NUCLEASE
gtcgcctatagggcgatacaactccgagcatgtgtcttccccttcaatgggcttggcactcggcatcgatcaagctcgcgtcggctgtcgggcc

SEQ ID NO: 22 - DNA, 111 bp, Artificial Sequence
  TEST SGRNA
gggaagggcctatttcccagcatgtgtcttcgcatttaattgctttagcactgggcatcgttctttttagtagtgtttaagtagatactactgaaaagaccgatggacaca

SEQ ID NO: 23 - DNA, 98 bp, Artificial Sequence
  TEST GRNA
gggaagggcctatttcccagcatgtgtcttcgcatttaattgctttagcactgggcatcgttctttttagaaaactactgaaaagaccgatggacaca

SEQ ID NO: 24 - DNA, 83 bp, Artificial Sequence
  TEST SGRNA
gggaagggcctatttcccagcatgtgtcttcgcatttaattgctttagcactgggcatcgttctaaaaagaccgatggacaca

SEQ ID NO: 25 - DNA, 97 bp, Artificial Sequence
  TEST SGRNA
ggaagggcctatttcccagcatgtgtcttcgcatttaattgctttagcactgggcatcgttctttttagaaaactactgaaaagaccgatggacaca

SEQ ID NO: 26 - DNA, 105 bp, Artificial Sequence
  TEST SGRNA
ggtccaagggaagggcctatttcccagcatgtgtcttcgcatttaattgctttagcactgggcatcgttctttttagaaaactactgaaaagaccgatggacaca

SEQ ID NO: 27 - DNA, 116 bp, Artificial Sequence
  TEST SGRNA
gccccgatttccctttgaatgatctcggcctcgttgccactgaccgaattcttccgcctttggaattccaagctctttgacatcgcgagcccgcgatgggaaagagattgtggcgg

SEQ ID NO: 28 - DNA, 114 bp, Artificial Sequence
  TEST SGRNA
gccccgatttccctttgaatgatctcggcctcgttgccactgaccgaattcttccgcctttggaattccaagctctttgacatcgcgagcccgcgatgggaaagagattgtggc

SEQ ID NO: 29 - DNA, 111 bp, Artificial Sequence
  TEST SGRNA
gatttccctttgaatgatctcggcctcgttgccactgaccgaattcttccgcctttggaattccaagctctttgacatcgcgagcccgcgatgggaaagagattgtggcgg

SEQ ID NO: 30 - DNA, 112 bp, Artificial Sequence
  TEST SGRNAV4
ggatttccctttgaatgatctcggcctcgttgccactgaccgaattcttccgcctttggaattccaagctctttgacatcgcgagcccgcgatgggaaagagattgtggcgg

SEQ ID NO: 31 - DNA, 110 bp, Artificial Sequence
  TEST SGRNA
gggaagggcctatttcccagcatgtgtcttcgcatttaattgctttagcactgggcatcgttctttttagtagtgtttaagtagatactactaaaaagaccgatggacac

SEQ ID NO: 32 - DNA, 96 bp, Artificial Sequence
  TEST SGRNA
gggaagggcctatttcccagcatgtgtcttcgcatttaattgctttagcactgggcatcgttctttttagtaaaaactaaaaagaccgatggacac

SEQ ID NO: 33 - DNA, 84 bp, Artificial Sequence
  TEST SGRNA
gggaagggcctatttcccagcatgtgtcttcgcatttaattgctttagcactgggcatcgttcttaaaaaagaccgatggacac

SEQ ID NO: 34 - DNA, 103 bp, Artificial Sequence
  TEST SGRNA
gggccaagggaagggcctatttcccagcatgtgtcttcgcatttaattgctttagcactgggcatcgttctttttagtaaaaactaaaaagaccgatggacac

SEQ ID NO: 35 - DNA, 91 bp, Artificial Sequence
  TEST SGRNA
gggccaagggaagggcctatttcccagcatgtgtcttcgcatttaattgctttagcactgggcatcgttcttaaaaaagaccgatggacac

SEQ ID NO: 36 - DNA, 134 bp, Artificial Sequence
  TEST SGRNA
atgtaaccctataggggttgcgtgagtcggccatagtgcctcggcaacagcgtaaaaaactgctgccagtggtcgaagtaagtcaacaaaacggaggtggtgaagtcacccccgttttgtaggcctactggcac

SEQ ID NO: 37 - DNA, 111 bp, Artificial Sequence
  TEST SGRNA
ggatgtaaccctataggggttgcgtgagtcggccatagtgcctcggcaacagcgtaaaaaactgctgccagtggtcgaagtaagtcaacaaaaatgtaggcctactggcac

SEQ ID NO: 38 - DNA, 108 bp, Artificial Sequence
  TEST SGRNA
ggtaaccctataggggttgcgtgagtcggccatagtgcctcggcaacagcgtaaaaaactgctgccagtggtcgaagtaagtcaacaaaaatgtaggcctactggcac

SEQ ID NO: 39 - DNA, 112 bp, Artificial Sequence
  TEST SGRNA
gggatgtaaccctataggggttgcgtgagtcggccatagtgcctcggcaacagcgtaaaaaactgctgccagtggtcgaagtaagtcaacaaaaatgtaggcctactggcac

SEQ ID NO: 40 - DNA, 109 bp, Artificial Sequence
  TEST SGRNA
gggtaaccctataggggttgcgtgagtcggccatagtgcctcggcaacagcgtaaaaaactgctgccagtggtcgaagtaagtcaacaaaaatgtaggcctactggcac

SEQ ID NO: 41 - DNA, 109 bp, Artificial Sequence
  TEST SGRNA
cggagggaactccgtgaacgtgtcttccccttcgatgggcttggcacacggggtcaatcgagttgccgttgaaggcggcgaattcgccggcatctcgatcgaccgacac

SEQ ID NO: 42 - DNA, 91 bp, Artificial Sequence
  TEST SGRNA
ggagggaactccgtgaacgtgtcttccccttcgatgggcttggcacacggggtcaatcgagttgccaaaaggcatctcgatcgaccgacac

SEQ ID NO: 43 - DNA, 81 bp, Artificial Sequence
  TEST SGRNA
ggagggaactccgtgaacgtgtcttccccttcgatgggcttggcacacggggtcaatcgagttatctcgatcgaccgacac

SEQ ID NO: 44 - DNA, 98 bp, Artificial Sequence
  TEST SGRNA
ggcaggcggagggaactccgtgaacgtgtcttccccttcgatgggcttggcacacggggtcaatcgagttgccaaaaggcatctcgatcgaccgacac

SEQ ID NO: 45 - DNA, 88 bp, Artificial Sequence
  TEST SGRNA
ggcaggcggagggaactccgtgaacgtgtcttccccttcgatgggcttggcacacggggtcaatcgagttatctcgatcgaccgacac

SEQ ID NO: 46 - DNA, 129 bp, Artificial Sequence
  TEST SGRNA
ttgcctctacaggaggcgagaatgccacggcacgtgtcttccccttcaatgggcttggcaccgtggagtcgatcagttttgtgccggcgaagggttgccgacgtcagcaccaacctgatcgacggacac

SEQ ID NO: 47 - DNA, 93 bp, Artificial Sequence
  TEST SGRNA
gcctctacaggaggcgagaatgccacggcacgtgtcttccccttcaatgggcttggcaccgtggagtcgatcagaaaactgatcgacggacac

SEQ ID NO: 48 - DNA, 95 bp, Artificial Sequence
  TEST SGRNA
gggcctctacaggaggcgagaatgccacggcacgtgtcttccccttcaatgggcttggcaccgtggagtcgatcagaaaactgatcgacggacac

SEQ ID NO: 49 - DNA, 102 bp, Artificial Sequence
  TEST SGRNA
gggacattggcctctacaggaggcgagaatgccacggcacgtgtcttccccttcaatgggcttggcaccgtggagtcgatcagaaaactgatcgacggacac

SEQ ID NO: 50 - DNA, 133 bp, Artificial Sequence
  TEST SGRNA
gtcgcctatagggcgatacaactccgagcatgtgtcttccccttcaatgggcttggcactcggcatcgatcaagctcgcgtcggctgtcgggccaaagccgcgtcggccgacgcggccttgatcgatggacac

SEQ ID NO: 51 - DNA, 120 bp, Artificial Sequence
  TEST SGRNA
gtcgcctatagggcgatacaactccgagcatgtgtcttccccttcaatgggcttggcactcggcatcgatcaagctcgcgtcggctgtcgtcggccgacgcggccttgatcgatggacac

SEQ ID NO: 52 - DNA, 108 bp, Artificial Sequence
  TEST SGRNA
gtcgcctatagggcgatacaactccgagcatgtgtcttccccttcaatgggcttggcactcggcatcgatcaagctcgcgtaaaaacgcggccttgatcgatggacac

SEQ ID NO: 53 - DNA, 121 bp, Artificial Sequence
  TEST SGRNA
ggtcgcctatagggcgatacaactccgagcatgtgtcttccccttcaatgggcttggcactcggcatcgatcaagctcgcgtcggctgtcgtcggccgacgcggccttgatcgatggacac

SEQ ID NO: 54 - DNA, 110 bp, Artificial Sequence
  TEST SGRNA
gggtcgcctatagggcgatacaactccgagcatgtgtcttccccttcaatgggcttggcactcggcatcgatcaagctcgcgtaaaaacgcggccttgatcgatggacac

SEQ ID NO: 55 - DNA, 20 bp, Artificial Sequence
  PRIMER SEQUENCE
ttgggtaacgccagggtttt

SEQ ID NO: 56 - DNA, 20 bp, Artificial Sequence
  PRIMER SEQUENCE
tgtgtggaattgtgagcgga

SEQ ID NO: 57 - DNA, 20 bp, Artificial Sequence
  PRIMER SEQUENCE
aaacccctccgtttagagag

SEQ ID NO: 58 - DNA, 29 bp, Artificial Sequence
  PRIMER SEQUENCE
aagctaatacgactcactataggccagtc

SEQ ID NO: 59 - DNA, 20 bp, Artificial Sequence
  PRIMER SEQUENCE
ccagtcagtaatgttactgg

SEQ ID NO: 60 - DNA, 23 bp, Artificial Sequence
  PRIMER SEQUENCE
ccggtggtgcagatgaacttcag

SEQ ID NO: 61 - DNA, 23 bp, Artificial Sequence
  PRIMER SEQUENCE
aagaagtcgtgctgcttcatgtg

SEQ ID NO: 62 - DNA, 23 bp, Artificial Sequence
  PRIMER SEQUENCE
ccgtaggtggcatcgccctcgcc

SEQ ID NO: 63 - DNA, 23 bp, Artificial Sequence
  PRIMER SEQUENCE
gggcatggcggacttgaagaagt

SEQ ID NO: 64 - DNA, 23 bp, Artificial Sequence
  PRIMER SEQUENCE
tggccgtttacgtcgccgtccag

SEQ ID NO: 65 - DNA, 23 bp, Artificial Sequence
  PRIMER SEQUENCE
aagaagatggtgcgctcctggac

SEQ ID NO: 66 - DNA, 23 bp, Artificial Sequence
  PRIMER SEQUENCE
gccggtggtgcagatgaacttca

SEQ ID NO: 67 - DNA, 23 bp, Artificial Sequence
  PRIMER SEQUENCE
atctgcaccaccggcaaactgcc

SEQ ID NO: 68 - DNA, 23 bp, Artificial Sequence
  PRIMER SEQUENCE
agcgtgtccggcgagggcgaggg

SEQ ID NO: 69 - DNA, 23 bp, Artificial Sequence
  PRIMER SEQUENCE
gtgaccaccctgacctacggcgt

SEQ ID NO: 70 - DNA, 23 bp, Artificial Sequence
  PRIMER SEQUENCE
agccgctaccccgaccacatgaa

SEQ ID NO: 71 - DNA, 23 bp, Artificial Sequence
  PRIMER SEQUENCE
ttcaagtccgccatgcccgaagg

SEQ ID NO: 72 - DNA, 23 bp, Artificial Sequence
  PRIMER SEQUENCE
ttcaaggacgacggcaactacaa

SEQ ID NO: 73 - DNA, 23 bp, Artificial Sequence
  PRIMER SEQUENCE
aaggacgacggcaactacaagac